SEQUENCE LISTING (19 sequences)

SEQ ID NO: 1 - RNA, 22 nt, synthetic construct
  source 1..22: mol_type = other RNA
tagcagcacataatggtttgtg

SEQ ID NO: 2 - DNA, 17 bp, synthetic construct
  source 1..17: mol_type = other DNA
ctcgcttcggcagcaca

SEQ ID NO: 3 - DNA, 19 bp, synthetic construct
  source 1..19: mol_type = other DNA
aacgttcacgaatttgcgt

SEQ ID NO: 4 - DNA, 30 bp, synthetic construct
  source 1..30: mol_type = other DNA
acactccagctgggtcaccgggtgtaaatc

SEQ ID NO: 5 - DNA, 44 bp, synthetic construct
  source 1..44: mol_type = other DNA
ctcaactggtgtcgtggagtcggcaattcagttgagcaagctga

SEQ ID NO: 6 - RNA, 22 nt, synthetic construct
  source 1..22: mol_type = other RNA
cacaaaccattatgtgctgcta

SEQ ID NO: 7 - RNA, 24 nt, synthetic construct
  source 1..24: mol_type = other RNA
  modified_base 23..24: mod_base = OTHER; note = thymine
tagcagcacataatggtttgtgtt

SEQ ID NO: 8 - RNA, 24 nt, synthetic construct
  source 1..24: mol_type = other RNA
  modified_base 23..24: mod_base = OTHER; note = thymine
cacaaaccattatgtgctgctatt

SEQ ID NO: 9 - DNA, 22 bp, synthetic construct
  source 1..22: mol_type = other DNA
tagcagcacataatggtttgtg

SEQ ID NO: 10-11 - skipped (2 entries)

SEQ ID NO: 12 - RNA, 22 nt, synthetic construct
  source 1..22: mol_type = other RNA
tagcagcacatgatggtttgtg

SEQ ID NO: 13 - RNA, 22 nt, synthetic construct
  source 1..22: mol_type = other RNA
tagcagcacataatagtttgtg

SEQ ID NO: 14 - RNA, 22 nt, synthetic construct
  source 1..22: mol_type = other RNA
tagcagcacataatggtttatg

SEQ ID NO: 15 - RNA, 22 nt, synthetic construct
  source 1..22: mol_type = other RNA
tagcagcacataatggttgatg

SEQ ID NO: 16 - RNA, 22 nt, synthetic construct
  source 1..22: mol_type = other RNA
tagcagcacataatggcatgtg

SEQ ID NO: 17 - RNA, 22 nt, synthetic construct
  source 1..22: mol_type = other RNA
tagcagcaagaaatggtttgtg

SEQ ID NO: 18 - RNA, 22 nt, synthetic construct
  source 1..22: mol_type = other RNA
tagcagcacataaaattttgtg

SEQ ID NO: 19 - RNA, 22 nt, synthetic construct
  source 1..22: mol_type = other RNA
tagcagcacataatggtccagt

(19) United States
(12) Patent Application Publication
(10) Pub. No.: US 2026/0125674 A1
(43) Pub. Date: May 7, 2026

(54) USE OF MIR-15A-5P IN TREATMENT OF FUNDUS OCULI DISEASE

(71) Applicant: TIANJIN MEDICAL UNIVERSITY EYE HOSPITAL, Tianjin (CN)

(72) Inventors: Xiaorong LI, Tianjin (CN); Xiaomin ZHANG, Tianjin (CN); Hui ZHANG, Tianjin (CN); Xinyue YU, Tianjin (CN)

(21) Appl. No.: 19/118,267

(22) PCT Filed: Jan. 23, 2024
(86) PCT No.: PCT/CN2024/073634

§ 371 Date: Apr. 3, 2025

(30) Foreign Application Priority Data
Jun. 21, 2023 (CN) 202310741893.4

(51) Int. Cl. C12N 15/113; A61K 9/00; A61K 31/7115; A61K 31/713; A61P 27/02; C12N 15/85

(52) U.S. Cl. CPC C12N 15/113; A61K 9/0048; A61K 31/7115; A61K 31/713; A61P 27/02; C12N 15/85; C12N 2310/11; C12N 2310/141; C12N 2310/3515

(57) ABSTRACT

The present disclosure relates to the technical field of biomedicine, and in particular to use of miRNA or modified miRNA in treating fundus oculi diseases. The miRNA or modified miRNA has biological activity, can be absorbed by cells in vitro or retina cells in vivo, and quickly plays a biological role. Through a topical administration mode, the miRNA or the modified miRNA can inhibit pathological neovascularization, promote recovery of a non-perfusion region, reduce expression of retinal inflammatory factors, relieve retinal fibrosis, and promote nerve injury repair. The method has very good application and research values in the field of biomedicine.

### TECHNICAL FIELD

The present disclosure relates to the technical field of biomedicine, and in particular to use of miRNA or modified miRNA (particularly miR-15a-5p or modified miR-15a-5p) in treating fundus oculi diseases.

### BACKGROUND

The fundus oculi is the most important physiological structure of the eyeball, including the choroid, retina, vitreous body, and the like. The retina, which is dotted with vasculatures and nerves, is the main structure responsible for visual functions and plays an important physiological role. Pathogenic factors such as elevated blood glucose levels or hypoxia can lead to retinal damage, resulting in vision loss. For example, retinopathy of prematurity (ROP) is a blinding fundus oculi disease commonly seen in preterm infants, and its pathogenesis involves retinal vascular degeneration due to neonatal oxygen intake and continuous massive neovascularization. The vascular abnormalities during neonatal retinal development also lead to neurodegeneration and degeneration, which greatly affects retinal development in infants. The current treatment is intravitreal injection of anti-vascular endothelial growth factor (VEGF) monoclonal antibodies, but it is a single-targeted therapy that fails to improve neurodegeneration in the retina and reduce retinal inflammation. Moreover, since VEGF is an important neurotrophic factor, the use of anti-VEGF therapy may lead to further damage of retinal nerve cells and decreased retinal function. Diabetic retinopathy (DR), which has a high incidence rate among the working-age population, is also a microvascular disease characterized primarily by neovascularization and neurodegeneration. Its pathogenic factor is continuously elevated blood glucose levels, but the subsequent pathogenesis is more complex. The current treatment is ablating peripheral retinal areas in the non-proliferative phase by laser photocoagulation to reduce vascular leakage and retinal oxygen consumption, but the laser therapy is somewhat destructive and cannot completely prevent the development of neovascularization. In the proliferative phase, neovascularization is usually inhibited by intravitreal injection of anti-VEGF monoclonal antibodies or vitrectomy. As previously mentioned, anti-VEGF therapy fails to ameliorate retinal nerve injury and function.

MicroRNA (miRNA) is a class of non-coding single-stranded RNA molecules encoded by endogenous genes with a length of about 22 nucleotides, which is involved in post-transcriptional gene expression regulation in plants and animals. miRNA is mainly characterized by its natural presence in the human body, multi-targeted regulation, and rich biological functions. miR-15a-5p is closely associated with the occurrence and development of various diseases.

Patent document CN112575088B discloses use of a plasma exosome miRNA biomarker in preparing a kit for screening and diagnosing endometrial cancer, and the biomarker is a combination of miR-15a-5p, miR-106b-5p, and miR-107.

Patent document CN109414459B discloses that exosomes comprising miRNA, e.g., miR-15a-5p, can promote wound healing.

Patent document CN113943800A discloses use of a reagent for detecting exosome miR-15a-5p in preparing a thyroid carcinoma iodine resistance screening kit.

Patent document CN110205377A discloses a method for evaluating the risk of Kawasaki disease in advance based on miRNA molecular markers, and the miRNA molecular markers are miR-30c-5p, miR-26a-5p, miR-27a-3p, miR-15a-5p, miR-186-5p, let-7g-5p, miR-941, miR-92a-3p, miR-22-3p, miR-151a-3p, miR-140-3p, miR-199a-3p, and miR-4433b-5p.

Currently, there is less application and mechanism research on miR-15a-5p with a single biological component in fundus oculi diseases in the prior art.

### SUMMARY

To overcome the defects in the prior art, the present application demonstrates the effectiveness of miRNA in treating fundus oculi diseases, and particularly, miR-15a-5p or modified miR-15a-5p has biological activity, can be absorbed by cells in vitro or retina cells in vivo, and quickly plays a biological role. Through a topical administration mode, the miR-15a-5p or the modified miR-15a-5p can inhibit pathological neovascularization, promote recovery of a non-perfusion region, reduce expression of retinal inflammatory factors, relieve retinal fibrosis, and promote nerve injury repair. In addition, it is experimentally determined that the active fragment of the miRNA is ACGACGAU (SEQ ID NO: 10), and the mutated or modified miR-15a-5p still exhibits good efficacy. The achievement has very good application and research values in the field of biomedicine.

The specific scheme is as follows:

In a first aspect of the present disclosure, provided is use of miRNA and/or modified miRNA and/or a vector comprising a nucleic acid to be transcribed into miRNA in preparing a product for treating and/or preventing a fundus oculi disease.

A nucleotide sequence of the miRNA comprises ACGACGAU (SEQ ID NO: 10).

Preferably, the nucleotide sequence of the miRNA comprises SEQ ID NO: 10, and has 60%, 65%, 70%, 75%, 76%, 77%, 78%, 79%, 80%, 81%, 82%, 83%, 84%, 85%, 86%, 87%, 88%, 89%, 90%, 95%, or 99% or more identity to the nucleotide sequence set forth in SEQ ID NO: 1.

Preferably, the nucleotide sequence of the miRNA comprises SEQ ID NO: 10, and comprises a nucleotide sequence with substitution, deletion, or insertion of one or more nucleotides as compared to the nucleotide sequence set forth in SEQ ID NO: 1, preferably a nucleotide sequence with substitution, deletion, or insertion of no more than ten, nine, eight, seven, six, five, four, three, two, or one nucleotide.

Preferably, the miRNA is miR-15a-5p.

In one specific embodiment of the present disclosure, the nucleotide sequence of the miRNA is SEQ ID NO: 1.

The fundus oculi disease is a fundus oculi disease which is beneficially prevented and/or treated by inhibiting VEGF and/or TGF-β1. Preferably, the simultaneous inhibition of VEGF and TGF-β1 is beneficial in the prevention and/or treatment of a fundus oculi disease.

The fundus oculi disease is selected from one or a combination of two or more of vitreous disease, retinopathy, optic neuropathy, or choroidopathy.

Preferably, the fundus oculi disease is selected from one or a combination of two or more of retinopathy of prematurity, retinal neovascular disease, choroidal neovascular disease, or diabetic retinopathy.

The miRNA and/or the modified miRNA and/or the vector comprising a nucleic acid to be transcribed into miRNA and/or targets and/or regulates VEGF and/or TGF-β1, and preferably targets VEGF and TGF-β1 simultaneously. The regulation is up-regulation or down-regulation. Preferably, the regulation of TGF-β1 comprises inhibiting Smad2 or is performed by inhibiting Smad2. Preferably, the regulation of VEGF comprises inhibiting VEGF.

Preferably, the miRNA and/or the modified miRNA and/or the vector comprising a nucleic acid to be transcribed into miRNA comprises a domain targeting VEGF and/or TGF-β1 (e.g., SEQ ID NO: 10).

Preferably, the sequence of the miRNA comprises a modification, e.g., a modification on a base.

SEQ ID NO: 1 comprises or is UAGCAGCACAUAAUGGUUUGUG.

The miRNA or the mimic thereof comprises a sense strand and an antisense strand. The sense strand comprises or is SEQ ID NO: 1, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 1, or comprises a nucleotide sequence with substitution, deletion, or insertion of one or more nucleotides as compared to the nucleotide sequence set forth in SEQ ID NO:1; and the antisense strand comprises or is CACAAACCAUUAUGUGCUGCUA (SEQ ID NO: 6), or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 6, or comprises a nucleotide sequence with substitution, deletion, or insertion of one or more nucleotides as compared to the nucleotide sequence set forth in SEQ ID NO:6. The product comprises miRNA and/or modified miRNA and/or a miRNA mimic, or comprises a vector comprising the miRNA and/or the modified miRNA and/or the miRNA mimic (the vector may be any vector that encapsulates or is transcribed into or expresses miRNA).

The vector is a viral vector or a non-viral vector.

Preferably, the viral vector comprises one or a combination of two or more of lentivirus vector, retrovirus vector, adenovirus vector, adeno-associated virus vector, poxvirus vector, or herpesvirus vector.

Preferably, the non-viral vector comprises one or a combination of two or more of liposome, lipid nanoparticle, polymer, polypeptide, antibody, aptamer, or N-acetylgalactosamine.

The sequence of the miRNA comprises a modification, e.g., a modification on a base.

The modified miRNA comprises a modification on a base.

The modification on the base is located at the sense strand and/or the antisense strand.

Preferably, the modification on the base comprises one or a combination of two or more of a cholesterol modification at the 3′ end, two thio-backbone modifications at the 5′ end, four thio-backbone modifications at the 3′ end, or a full-chain methoxy modification.

The product comprises a sense strand and an antisense strand.

Preferably, the sense strand and/or the antisense strand comprises an over-hang.

The over-hang is located at the 3′ end of the sense strand and/or the antisense strand.

The over-hang is deoxynucleoside. Preferably, the over-hang is dTdT, dTdC, or dUdU. For example, the miRNA or the mimic thereof may comprise a sense strand of UAGCAGCACAUAAUGGUUUGUGdTdT (SEQ ID NO: 7) and an antisense strand of CACAAACCAUUAUGUGCUGCUAdTdT (SEQ ID NO: 8).

The sense strand and/or the antisense strand comprised in the miRNA or the mimic thereof is subjected to one or a combination of two or more of a full-chain methoxy modification, a cholesterol modification at the 3′ end, a thio-backbone modification at the 5′ end, or a thio-backbone modification at the 3′ end.

In one specific embodiment of the present disclosure, the antisense strand of the miRNA or the mimic thereof is subjected to a full-chain methoxy modification, a cholesterol modification at the 3′ end, two thio-backbone modifications at the 5′ end, and four thio-backbone modifications at the 3′ end.

In one specific embodiment of the present disclosure, the sense strand comprises or is SEQ ID NO: 1, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 1, or comprises a nucleotide sequence with substitution, deletion, or insertion of one or more nucleotides as compared to the nucleotide sequence set forth in SEQ ID NO: 1; and the antisense strand comprises SEQ ID NO: 6 with two thio-backbone modifications at the 5′ end, four thio-backbone modifications at the 3′ end, a cholesterol modification at the 3′ end, and a full-chain methoxy modification, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 6, or comprises a nucleotide sequence with substitution, deletion, or insertion of one or more nucleotides as compared to the nucleotide sequence set forth in SEQ ID NO: 6.

The treating and/or preventing the fundus oculi disease is selected from one or a combination of two or more of inhibiting the activity of human retinal microvascular endothelial cells (HRMECs), inhibiting the proliferation of the HRMECs (particularly weakening the proliferation of HRMECs induced by VEGF), inhibiting the phosphorylation of Smad2, inhibiting the expression of total Smad2, inhibiting fibrosis, inhibiting the expression of VEGF, inhibiting the inflammation of retina, resisting neovascularization or promoting the recovery of a non-perfusion region, ameliorating the thinning of the retina, recovering the visual function, or promoting nerve injury repair.

Preferably, the resisting neovascularization comprises inhibiting retinal neovascularization and/or choroidal neovascularization.

The treating and/or preventing the fundus oculi disease comprises administering to a subject in need thereof miRNA and/or modified miRNA or a miRNA mimic, and/or administering a vector comprising the miRNA and/or the modified miRNA or the miRNA mimic (e.g., a vector comprising a nucleic acid to be transcribed into miRNA).

A site of the administration may be an intraocular space or cavity of the subject, e.g., one or a combination of two or more of aqueous humor in the anterior chamber, suspensory ligament, ciliary body, internal ciliary body and muscle, lens or iris, vitreous body, retina, choroid, or optic nerve.

In a second aspect of the present disclosure, provided is a vector comprising a nucleic acid to be transcribed into miRNA or a mimic thereof.

The nucleotide sequence of the miRNA comprises SEQ ID NO: 10.

Preferably, the nucleotide sequence of the miRNA comprises SEQ ID NO: 10, and has 60%, 65%, 70%, 75%, 76%, 77%, 78%, 79%, 80%, 81%, 82%, 83%, 84%, 85%, 86%, 87%, 88%, 89%, 90%, 95%, or 99% or more identity to the nucleotide sequence set forth in SEQ ID NO: 1.

Preferably, the nucleotide sequence of the miRNA comprises SEQ ID NO: 10, and comprises a nucleotide sequence with substitution, deletion, or insertion of one or more nucleotides as compared to the nucleotide sequence set forth in SEQ ID NO: 1, preferably a nucleotide sequence with substitution, deletion, or insertion of no more than ten, nine, eight, seven, six, five, four, three, two, or one nucleotide.

Preferably, the miRNA is miR-15a-5p.

In one specific embodiment of the present disclosure, the nucleotide sequence of the miRNA is SEQ ID NO: 1.

The sequence of the miRNA comprises a modification, e.g., a modification on a base. The modification on the base is located at the sense strand and/or the antisense strand, and preferably comprises one or a combination of two or more of a cholesterol modification at the 3′ end, two thio-backbone modifications at the 5′ end, four thio-backbone modifications at the 3′ end, or a full-chain methoxy modification.

The nucleic acid to be transcribed into miRNA comprises TAGCAGCA (SEQ ID NO: 11).

Preferably, the nucleic acid to be transcribed into miRNA comprises SEQ ID NO: 11, and has 60%, 65%, 70%, 75%, 76%, 77%, 78%, 79%, 80%, 81%, 82%, 83%, 84%, 85%, 86%, 87%, 88%, 89%, 90%, 95%, or 99% or more identity to the nucleotide sequence set forth in SEQ ID NO: 9.

Preferably, the nucleic acid to be transcribed into miRNA comprises SEQ ID NO: 11, and comprises a nucleotide sequence with substitution, deletion, or insertion of one or more nucleotides as compared to the nucleotide sequence set forth in SEQ ID NO: 9, preferably a nucleotide sequence with substitution, deletion, or insertion of no more than ten, nine, eight, seven, six, five, four, three, two, or one nucleotide.

In one specific embodiment of the present disclosure, the nucleic acid to be transcribed into miRNA is SEQ ID NO: 9.

The miRNA or the mimic thereof comprises a sense strand and an antisense strand. The sense strand comprises SEQ ID NO: 1, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 1, or comprises a nucleotide sequence with substitution, deletion, or insertion of one or more nucleotides as compared to the nucleotide sequence set forth in SEQ ID NO:1; and the antisense strand comprises SEQ ID NO: 6, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 6, or comprises a nucleotide sequence with substitution, deletion, or insertion of one or more nucleotides as compared to the nucleotide sequence set forth in SEQ ID NO:6.

The sense strand and/or the antisense strand comprises an over-hang.

The over-hang is located at the 3′ end of the sense strand and/or the antisense strand.

The over-hang is deoxynucleoside.

The over-hang is dTdT, dTdC, or dUdU.

The vector is a viral vector or a non-viral vector.

The viral vector comprises one or a combination of two or more of lentivirus vector, retrovirus vector, adenovirus vector, adeno-associated virus vector, poxvirus vector, or herpesvirus vector.

The non-viral vector comprises one or a combination of two or more of liposome, lipid nanoparticle, polymer, polypeptide, antibody, aptamer, or N-acetylgalactosamine.

In a third aspect of the present disclosure, provided is a modified miR-15a-5p or a miR-15a-5p mimic.

The modified miR-15a-5p comprises a modification on a base.

The modification on the base is located at the antisense strand. Preferably, the modification comprises one or a combination of two or more of a cholesterol modification at the 3′ end, two thio-backbone modifications at the 5′ end, four thio-backbone modifications at the 3′ end, or a full-chain methoxy modification.

The sense strand and/or the antisense strand comprised in the miR-15a-5p or the mimic thereof is subjected to one or a combination of two or more of a full-chain methoxy modification, a cholesterol modification at the 3′ end, a thio-backbone modification at the 5′ end, or a thio-backbone modification at the 3′ end.

In one specific embodiment of the present disclosure, the antisense strand of the miR-15a-5p or the mimic thereof is subjected to a full-chain methoxy modification, a cholesterol modification at the 3′ end, two thio-backbone modifications at the 5′ end, and four thio-backbone modifications at the 3′ end.

In one specific embodiment of the present disclosure, the sense strand comprises SEQ ID NO: 1, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 1, or comprises a nucleotide sequence with substitution, deletion, or insertion of one or more nucleotides as compared to the nucleotide sequence set forth in SEQ ID NO: 1; and the antisense strand comprises SEQ ID NO: 6 with two thio-backbone modifications at the 5′ end, four thio-backbone modifications at the 3′ end, a cholesterol modification at the 3′ end, and a full-chain methoxy modification, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 6, or comprises a nucleotide sequence with substitution, deletion, or insertion of one or more nucleotides as compared to the nucleotide sequence set forth in SEQ ID NO: 6.

In a fourth aspect of the present disclosure, provided is a medicament or a pharmaceutical composition. The medicament or the pharmaceutical composition comprises the miRNA and/or the modified miRNA and/or the miRNA mimic described above and/or the vector comprising the nucleic acid to be transcribed into the miRNA or the miRNA mimic described above, and a pharmaceutically acceptable auxiliary material.

A nucleotide sequence of the miRNA comprises ACGACGAU (SEQ ID NO: 10).

Preferably, the nucleotide sequence of the miRNA comprises SEQ ID NO: 10 and has 60%, 65%, 70%, 75%, 76%, 77%, 78%, 79%, 80%, 81%, 82%, 83%, 84%, 85%, 86%, 87%, 88%, 89%, 90%, 95%, or 99% or more identity to the nucleotide sequence set forth in SEQ ID NO: 1.

Preferably, the nucleotide sequence of the miRNA comprises SEQ ID NO: 10, and comprises a nucleotide sequence with substitution, deletion, or insertion of one or more nucleotides as compared to the nucleotide sequence set forth in SEQ ID NO: 1, preferably a nucleotide sequence with substitution, deletion, or insertion of no more than ten, nine, eight, seven, six, five, four, three, two, or one nucleotide.

Preferably, the miRNA is miR-15a-5p.

In one specific embodiment of the present disclosure, the nucleotide sequence of the miRNA is SEQ ID NO: 1.

The modified miRNA comprises a modification on a base.

Preferably, the modification on the base comprises one or a combination of two or more of a cholesterol modification at the 3′ end, two thio-backbone modifications at the 5′ end, four thio-backbone modifications at the 3′ end, or a full-chain methoxy modification.

The medicament or the pharmaceutical composition can treat fundus oculi diseases.

The pharmaceutical composition may further comprise other nucleic acids, polypeptides, proteins, compounds, and the like for treating or preventing fundus oculi diseases, or nucleic acids, polypeptides, proteins, compounds, and the like for reducing side effects.

The medicament or the pharmaceutical composition may be administered by any suitable route of administration, such as gastrointestinal routes (e.g., oral administration) or parenteral routes (e.g., intravenous, intramuscular, subcutaneous, intradermal, intraorgan, intranasal, intraocular, instillation, intracerebral, intrathecal, transdermal, or intrarectal administrations, and the like).

The medicament or the pharmaceutical composition may be in any suitable dosage form, such as dosage forms for gastrointestinal or parenteral administration. The dosage forms preferably include, but are not limited to, tablets, pills, powders, granules, capsules, lozenges, syrups, liquids, emulsions, microemulsions, suspensions, injections, sprays, aerosols, power aerosols, lotions, ointments, plasters, pastes, patches, eye drops, nose drops, sublingual tablets, suppositories, aerosols, effervescent tablets, drop pills, gels, and the like.

The dosage forms of the medicament or the pharmaceutical composition can be prepared according to conventional production methods in the field of pharmaceuticals.

The medicament or the pharmaceutical composition may comprise 0.01-99.5% (specifically, 0.01%, 0.1%, 0.5%, 1%, 2%, 3%, 4%, 5%, 6%, 7%, 8%, 9%, 10%, 20%, 30%, 40%, 50%, 60%, 70%, 80%, 90%, 95%, 99%, or 99.5%) by weight of the miRNA, the modified miRNA, or the vector comprising the miRNA or the modified miRNA.

The medicament or the pharmaceutical composition may be a human medicament or a veterinary medicament. In a fifth aspect of the present disclosure, provided is a method for treating and/or preventing a fundus oculi disease. The method comprises administering to a subject in need thereof an effective amount of miRNA and/or modified miRNA and/or a miRNA mimic, and/or a vector and/or a pharmaceutical composition comprising the miRNA or the modified miRNA or the miRNA mimic.

A nucleotide sequence of the miRNA comprises ACGACGAU (SEQ ID NO: 10).

Preferably, the nucleotide sequence of the miRNA comprises SEQ ID NO: 10, and has 60%, 65%, 70%, 75%, 76%, 77%, 78%, 79%, 80%, 81%, 82%, 83%, 84%, 85%, 86%, 87%, 88%, 89%, 90%, 95%, or 99% or more identity to the nucleotide sequence set forth in SEQ ID NO: 1.

Preferably, the nucleotide sequence of the miRNA comprises SEQ ID NO: 10, and comprises a nucleotide sequence with substitution, deletion, or insertion of one or more nucleotides as compared to the nucleotide sequence set forth in SEQ ID NO: 1, preferably a nucleotide sequence with substitution, deletion, or insertion of no more than ten, nine, eight, seven, six, five, four, three, two, or one nucleotide.

Preferably, the miRNA is miR-15a-5p.

In one specific embodiment of the present disclosure, the nucleotide sequence of the miRNA is SEQ ID NO: 1.

The fundus oculi disease is a fundus oculi disease which is beneficially prevented and/or treated by inhibiting VEGF and/or TGF-β1.

The fundus oculi disease is selected from one or a combination of two or more of vitreous disease, retinopathy, optic neuropathy, or choroidopathy.

The fundus oculi disease includes but is not limited to one or a combination of two or more of retinopathy of prematurity, retinal neovascular disease, choroidal neovascular disease, or diabetic retinopathy.

The miRNA or the mimic thereof comprises a sense strand and an antisense strand. The sense strand comprises SEQ ID NO: 1, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 1, or comprises a nucleotide sequence with substitution, deletion, or insertion of one or more nucleotides as compared to the nucleotide sequence set forth in SEQ ID NO: 1; and the antisense strand comprises SEQ ID NO: 6, or has 60%, 65%, 70%, 75%, 76%, 77%, 78%, 79%, 80%, 81%, 82%, 83%, 84%, 85%, 86%, 87%, 88%, 89%, 90%, 95%, or 99% or more homology to the nucleotide sequence set forth in SEQ ID NO: 6, or comprises a nucleotide sequence with substitution, deletion, or insertion of one or more nucleotides as compared to the nucleotide sequence set forth in SEQ ID NO:6.

The modified miRNA comprises a modification on a base. The modification on the base comprises one or a combination of two or more of a cholesterol modification at the 3′ end, two thio-backbone modifications at the 5′ end, four thio-backbone modifications at the 3′ end, or a full-chain methoxy modification.

The treating and/or preventing the fundus oculi disease is selected from one or a combination of two or more of inhibiting the activity of HRMECs, inhibiting the proliferation of the HRMECs (particularly weakening the proliferation of HRMECs induced by VEGF), inhibiting the phosphorylation of Smad2, inhibiting the expression of total Smad2, inhibiting fibrosis, inhibiting the expression of VEGF, inhibiting the inflammation of retina, resisting neovascularization or promoting the recovery of a non-perfusion region, ameliorating the thinning of the retina, recovering the visual function, or promoting nerve injury repair.

Preferably, the resisting neovascularization comprises inhibiting retinal neovascularization and/or choroidal neovascularization.

Preferably, the method comprises administering to each eye 0.5 μg-5 mg (e.g., 0.5 μg, 1 μg, 2 μg. 3 μg, 4 μg, 5 μg, 10 μg, 20 μg, 50 μg, 100 μg, 150 μg, 200 μg, 250 μg, 300 μg, 350 μg, 400 μg, 450 μg, 500 μg, 550 μg. 600 μg, 650 μg, 700 μg, 750 μg, 800 μg, 850 μg, 900 μg, 950 μg, 1 mg, 1.5 mg, 2 mg, 2.5 mg, 3 mg, 3.5 mg, 4 mg, 4.5 mg, or 5 mg) of miRNA, modified miRNA, a miRNA mimic, a vector comprising the miRNA or the modified miRNA or the miRNA mimic, or the medicament or the pharmaceutical composition described above. Preferably, the method comprises administering to an intraocular space or cavity of the subject, e.g., one or a combination of two or more of aqueous humor in the anterior chamber, suspensory ligament, ciliary body, internal ciliary body and muscle, lens or iris, vitreous body, retina, choroid, or optic nerve.

In vivo experimental validation of the modified miRNA (e.g., modified mir-15a-5p, with modifications on the antisense strand base including a cholesterol modification at the 3′ end, two thio-backbone modifications at the 5′ end, four thio-backbone modifications at the 3′ end, and a full-chain methoxy modification, i.e., Agomir-15a-5p) is compared to the clinically-classic anti-VEGF drug ranibizumab. The result shows that the effect of the modified miRNA on inhibiting retinal and choroidal neovascularization is comparable to that of ranibizumab, and it has the following four advantages over ranibizumab: firstly, the modified miRNA not only inhibits neovascularization but also promotes retinal blood flow recovery, reducing the area of retinal non-perfusion regions: secondly, the modified miRNA has a function of protecting optic nerves: thirdly, the modified miRNA has a function of inhibiting retinal inflammatory factors and inflammatory mediators; and fourthly, the modified miRNA has a function of reducing retinal fibrosis changes.

The term “pharmaceutically acceptable” described in the present disclosure refers to the biological activity and properties of neither significantly stimulating an organism nor inhibiting the active substance of the administered product.

The term “pharmaceutically acceptable auxiliary material” described in the present disclosure includes, but is not limited to, one or a combination of two or more of vectors, excipients, diluents, wetting agents, fillers, binders, lubricants, disintegrants, antioxidants, buffers, suspending agents, solubilizers, thickeners, stabilizers, flavoring agents, or preservatives.

The term “treating” described in the present disclosure refers to after the disease has begun to progress, slowing, interrupting, arresting, controlling, stopping, reducing, or reversing the progression or severity of a sign, symptom, disorder, condition, or disease, but does not necessarily involve the complete elimination of all disease-related signs, symptoms, conditions, or disorders.

The term “effective amount” described in the present disclosure refers to an amount or dose of the miRNA, the miRNA mimic, the modified miRNA, and the medicament or the pharmaceutical composition of the present disclosure which provides the desired treatment or prevention after being administered to an individual or organ in a single dose or multiple doses.

The term “preventing” described in the present disclosure refers to the practice of preventing or delaying the onset of a disease or condition or symptom in the body.

The term “subject” described in the present disclosure may be a human or non-human mammal, and the non-human mammal may be a wild animal, a zoo animal, an economic animal, a companion animal, a laboratory animal, and the like. Preferably, the non-human mammal includes, but is not limited to, a pig, a cow, a sheep, a horse, a donkey, a fox, a racoon dog, a mink, a camel, a dog, a cat, a rabbit, a murine (e.g., a rat, a mouse, a guinea pig, a hamster, a gerbil, a chinchilla, and a squirrel), a monkey, or the like.

### BRIEF DESCRIPTION OF THE DRAWINGS

Hereinafter, embodiments of the present disclosure will be described in detail with reference to the accompanying drawings, in which:

FIG. 1: Detection of expression level of miR-15a-5p in retinal microvascular endothelial cells after transfecting retinal microvascular endothelial cells with mimic control/mimic/inhibitor control/inhibitor of miR-15a-5p. respectively. The mimic can increase the expression level of miR-15a-5p in the cells, and the inhibitor can reduce the expression level of miR-15a-5p in the cells.

FIG. 2: Effect on cell proliferation by stimulating retinal microvascular endothelial cells with VEGF at different concentrations. VEGF at 10 ng/mL can induce abnormal proliferation of the cells.

FIG. 3: Effect on relative expression level of miR-15a-5p by stimulating retinal microvascular endothelial cells with VEGF at different concentrations. VEGF at 10 ng/mL does not affect the expression level of miR-15a-5p in the cells.

FIG. 4: Detection of effect of miR-15a-5p on cell proliferation activity with CCK-8 kit. The miR-15a-5p mimic weakens the VEGF-induced pathological proliferation of retinal microvascular endothelial cells.

FIG. 5: Detection of effect of miR-15a-5p on cell proliferation capacity by cell scratch assay.

FIG. 6: Cell scratch quantitative statistical analysis. The miR-15a-5p mimic reduces the VEGF-induced pathological proliferation of human retinal endothelial cells.

FIG. 7: Detection of effect of miR-15a-5p on cell migration ability by Transwell assay.

FIG. 8: Quantitative statistical analysis on number of cells migrated in Transwell assay. The miR-15a-5p mimic reduces the VEGF-induced pathological migration of retinal microvascular endothelial cells.

FIG. 9: Detection of effect of miR-15a-5p on in vitro tube formation ability of cells by tube formation assay.

FIG. 10: Quantitative statistical analysis of total length of vascular tube formed by cells in the tube formation assay. The miR-15a-5p mimic reduces the VEGF-induced vascular tube production of retinal microvascular endothelial cells.

FIG. 11: Quantitative statistical analysis of number of vascular tube nodes formed by cells in tube formation assay. The miR-15a-5p mimic reduces the VEGF-induced vascular tube production of retinal microvascular endothelial cells.

FIG. 12: Differences in uptake of modified (Agomir) and unmodified (mimic) miR-15a-5p mimics in mouse retinas. * indicates P<0.05 for the mimic group versus the control group, and ** indicates P<0.01: @ indicates P<0.05 for the modified mimic (Agomir) versus the control group, @@@ indicates P<0.001, and @@@@ indicates P<0.0001: #indicates P<0.05 for the mimic group versus the modified mimic (Agomir) group, and ####indicates P<0.0001.

FIG. 13: Flowchart of oxygen-induced mouse retinal neovascularization model. P represents the days after the birth of the mice.

FIG. 14: Expression level of miR-15a-5p in retinas during development process of oxygen-induced retinal neovascularization mice. Normoxia refers to untreated mice, and hyperoxia refers to the neovascularization modeling group.

FIG. 15: Effect of miR-15a-5p mimic at different doses on treating oxygen-induced retinal neovascularization. Normoxia refers to untreated mice, and hyperoxia refers to the neovascularization modeling group. The mimic is Agomir.

FIG. 16: Statistical chart of effect of miR-15a-5p mimic at different doses on treating oxygen-induced retinal neovascularization. Normoxia refers to untreated mice, and hyperoxia refers to the neovascularization modeling group. The mimic is Agomir, and the mimic control is scrambled Agomir.

FIG. 17: Effect of anti-VEGF drug at different doses on treating oxygen-induced retinal neovascularization. A is a staining image, and B is a statistical chart. Normoxia refers to untreated mice, and hyperoxia refers to the neovascularization modeling group.

FIG. 18: Distribution and duration of Agomir-15a-5p in retinas after administration. (A) Representative image of CY3-labeled Agomir-15a-5p in retinas. (B) Quantification of fluorescence intensity of CY3-labeled Agomir-15a-5p. (C) Expression trend of Agomir-15a-5p in OIR retina after injection.

FIG. 19: Schematic image of effect of 1 μg of miR-15a-5p mimic on treating oxygen-induced retinal neovascularization. Normoxia (column 1) refers to untreated mice, and hyperoxia (columns 2-4) refers to the neovascularization modeling group. The mimic is Agomir, the mimic control is scrambled Agomir, and the anti-VEGF is ranibizumab.

FIG. 20: Statistical chart of neovascular area of 1 μg of miR-15a-5p mimic in treating oxygen-induced retinal neovascularization. Normoxia refers to untreated mice, and hyperoxia refers to the neovascularization modeling group. The mimic is Agomir, the mimic control is scrambled Agomir, and the VEGF monoclonal antibody is ranibizumab.

FIG. 21: Statistical chart of non-perfusion region area of 1 μg of miR-15a-5p mimic in treating oxygen-induced retinal neovascularization. Normoxia refers to untreated mice, and hyperoxia refers to the neovascularization modeling group. The mimic is Agomir, the mimic control is scrambled Agomir, and the VEGF monoclonal antibody is ranibizumab.

FIG. 22: Schematic structure of adeno-associated virus comprising miR-15a-5p.

FIG. 23: Schematic image of infection of retina by adeno-associated virus comprising miR-15a-5p.

FIG. 24: PCR results showing retinal miR-15a-5p overexpression folds after adeno-associated virus infection of retina. OIR-AAV-NC is the control virus injection group, and OIR-AAV-15a is the adeno-associated virus comprising miR-15a-5p injection group.

FIG. 25: Schematic image of therapeutic effect of adeno-associated virus comprising miR-15a-5p on retinal neovascularization and non-perfusion region. OIR-AAV-NC is the control virus injection group, and OIR-AAV-15a is the adeno-associated virus comprising miR-15a-5p injection group.

FIG. 26: Statistics of therapeutic effect of adeno-associated virus comprising miR-15a-5p on non-perfusion region.

FIG. 27: Statistics of therapeutic effect of adeno-associated virus comprising miR-15a-5p on oxygen-induced retinal neovascularization.

FIG. 28: A: Magnified image of retinal non-perfusion region showing differences in activation of astrocytes and Müller glial cells (punctate staining). B: Representative image showing retinal tip cells and filopodia. Original magnification, ×40. C: Statistical analysis of retinal filopodia. (n=retinas of 6 mice). D: Representative image showing interaction between end-foot regions of tip cells, GFAP-positive astrocytes, and Müller cells. Normoxia refers to untreated mice, and hyperoxia refers to the neovascularization modeling group. The mimic is Agomir, the mimic control is scrambled Agomir, and the VEGF monoclonal antibody is ranibizumab.

FIG. 29: Schematic image of area of non-perfusion region and neovascularization of 1 μg of miR-15a-5p mimic in treating oxygen-induced retinal neovascularization. The hyperoxia-normal mouse refers to the normal mice neovascularization modeling group, and the hyperoxia-knockout mouse refers to miR-15a-5p knockout mice neovascularization modeling group.

FIG. 30: Statistical chart of non-perfusion region area of 1 μg of miR-15a-5p mimic in treating oxygen-induced retinal neovascularization. The hyperoxia-normal mouse refers to the normal mice neovascularization modeling group, and the hyperoxia-knockout mouse refers to miR-15a-5p knockout mice neovascularization modeling group.

FIG. 31: Statistical chart of neovascular cluster area of 1 μg of miR-15a-5p mimic in treating oxygen-induced retinal neovascularization. The hyperoxia-normal mouse refers to the normal mice neovascularization modeling group, and the hyperoxia-knockout mouse refers to miR-15a-5p knockout mice neovascularization modeling group.

FIG. 32: Schematic image of area of non-perfusion region and neovascularization of 1 μg of miR-15a-5p mimic in treating oxygen-induced retinal neovascularization. The hyperoxia-normal mouse refers to the normal mice neovascularization modeling group, and the hyperoxia-knockout mouse refers to miR-15a-5p knockout mice neovascularization modeling group. The mimic control is scrambled Agomir, and the mimic is Agomir.

FIG. 33: Statistical chart of non-perfusion region area of 1 μg of miR-15a-5p mimic in treating oxygen-induced retinal neovascularization. The normal refers to the normal mice neovascularization modeling group, and the knockout refers to miR-15a-5p knockout mice neovascularization modeling group. The mimic control is scrambled Agomir, and the mimic is Agomir.

FIG. 34: Statistical chart of neovascular cluster area of 1 μg of miR-15a-5p mimic in treating oxygen-induced retinal neovascularization. The normal refers to the normal mice neovascularization modeling group, and the knockout refers to miR-15a-5p knockout mice neovascularization modeling group. The mimic control is scrambled Agomir, and the mimic is Agomir.

FIG. 35: Magnified image of retinal non-perfusion region showing differences in activation of astrocytes and Müller glial cells (punctate staining). The hyperoxia-normal mouse refers to the normal mice neovascularization modeling group, and the hyperoxia-knockout mouse refers to miR-15a-5p knockout mice neovascularization modeling group. The mimic control is scrambled Agomir, and the mimic is Agomir.

FIG. 36: Representative image showing retinal tip cells and filopodia, as well as interaction between end-foot regions of tip cells, GFAP-positive astrocytes, and Müller cells. Original magnification, ×40. The hyperoxia-normal mouse refers to the normal mice neovascularization modeling group, and the hyperoxia-knockout mouse refers to miR-15a-5p knockout mice neovascularization modeling group. The mimic control is scrambled Agomir, and the mimic is Agomir.

FIG. 37: Flowchart of laser-induced choroidal neovascularization model.

FIG. 38: Effect of miR-15a-5p on inhibiting choroidal neovascularization observed by fundus fluorescein angiography (FFA).

FIG. 39: Results of quantitative analysis of fluorescence leakage.

FIG. 40: Choroidal flatmounts for IB4 staining to quantify neovascular cluster area and to observe effect of miR-15a-5p on inhibiting choroidal neovascularization. IB4 positive indicates neovascular clusters.

FIG. 41: Results of quantitative analysis of neovascular clusters.

FIG. 42: Schematic fluorescent image of infection of retina and choroid by adeno-associated virus comprising miR-15a-5p.

FIG. 43: PCR results showing retinochoroidal miR-15a-5p overexpression folds after adeno-associated virus infection of retina and choroid. CNV-AAV-NC is the group of choroidal neovascularization with control virus injection, and CNV-AAV-15a is the group of choroidal neovascularization with adeno-associated virus comprising miR-15a-5p injection.

FIG. 44: Schematic image of choroidal flatmounts for IB4 staining to quantify neovascular cluster area and to observe inhibition of choroidal neovascularization by adeno-associated virus comprising miR-15a-5p.

FIG. 45: Statistical chart of choroidal flatmounts for IB4 staining to quantify neovascular cluster area and to observe inhibition of choroidal neovascularization by adeno-associated virus comprising miR-15a-5p.

FIG. 46: Retinal H&E staining showing layered structure and cellular morphology of retina. The asterisk indicates the outer plexiform layer. GCL represents the ganglion cell layer, IPL represents the inner plexiform layer, INL represents the inner nuclear layer, OPL represents the outer plexiform layer, ONL represents the outer nuclear layer, and RPE represents the pigment epithelium layer. Normoxia represents untreated mice, and hyperoxia represents oxygen-induced retinal neovascularization model mice.

FIG. 47: Schematic image of thickness of retinal layers of mice on postnatal day 42 quantified by using optical coherence tomography (OCT). IPL represents the inner plexiform layer, INL represents the inner nuclear layer, OPL represents the outer plexiform layer, and ONL represents the outer nuclear layer. Normoxia represents untreated mice, and hyperoxia represents oxygen-induced retinal neovascularization model mice.

FIG. 48: Topographic map of thickness of retinal layers of mice on postnatal day 42 quantified by using optical coherence tomography (OCT). Normoxia represents untreated mice, and hyperoxia represents oxygen-induced retinal neovascularization model mice.

FIG. 49: Statistics of thickness of retinal layers of mice on postnatal day 42 quantified by using optical coherence tomography (OCT) to determine protective effect of miR-15a-5p on retinal structure. Normoxia represents untreated mice, and hyperoxia represents oxygen-induced retinal neovascularization model mice.

FIG. 50: Schematic diagram of retinal function analysis of mice on postnatal days 25 and 42 using electroretinography (ERG). Normoxia represents untreated mice, and hyperoxia represents oxygen-induced retinal neovascularization model mice.

FIG. 51: Statistical chart of retinal function analysis of mice on postnatal days 25 and 42 using electroretinography (ERG) to determine protective effect of miR-15a-5p on retinal function. Normoxia represents untreated mice, and hyperoxia represents oxygen-induced retinal neovascularization model mice.

FIG. 52: Retinochoroidal H&E staining showing layered structure and cellular morphology of retina and choroid. GCL represents the ganglion cell layer, IPL represents the inner plexiform layer, INL represents the inner nuclear layer, OPL represents the outer plexiform layer, ONL represents the outer nuclear layer, and RPE represents the pigment epithelium layer.

FIG. 53: Schematic diagram of retinal function analysis of choroidal neovascularization model using electroretinography (ERG). The mimic control is scrambled Agomir, the mimic is Agomir, and the VEGF monoclonal antibody is ranibizumab.

FIG. 54: Statistical chart of retinal function analysis of choroidal neovascularization model using electroretinography (ERG). The mimic control is scrambled Agomir, the mimic is Agomir, and the VEGF monoclonal antibody is ranibizumab.

FIG. 55: Inhibition of glial cell proliferation in OIR retina by miR-15a-5p. (A) Representative image of GFAP immunostaining in retinal sections of normoxic and hyperoxic mice. (B) Quantification and comparison of GFAP intensities of groups described above. (C) Western blots showing expression of GFAP in retinas of normoxic and hyperoxic mice. (D) Quantification of protein level of GFAP by densitometry with endogenous reference of GAPDH level.

FIG. 56: Detection of content of TNFα in retinas of mice with oxygen-induced retinopathy at different time points by enzyme-linked immunosorbent assay (ELISA).

FIG. 57: Schematic image of expression level of intercellular adhesion molecule 1 (ICAM-1) in retinas of mice with oxygen-induced retinopathy in groups by western blot.

FIG. 58: Statistics of expression level of intercellular adhesion molecule 1 (ICAM-1) in retinas of mice with oxygen-induced retinopathy in groups by western blot.

FIG. 59: PCR results showing laser-induced retinal inflammatory factor levels of mice in groups. FIG. A refers to TNFα level, FIG. B refers to ICAM-1 level, and FIG. C refers to IL-1ß level.

FIG. 60: PCR results showing that miR-15a-5p can reduce TGF-β1-induced VEGF mRNA level in RPE cells.

FIG. 61: Schematic image showing that miR-15a-5p can reduce TGF-β1-induced VEGF protein level increase in RPE cells by western blot.

FIG. 62: Statistics of miR-15a-5p showing that miR-15a-5p can reduce TGF-β1-induced VEGF protein level increase in RPE cells by western blot.

FIG. 63: miR-15a-5p reducing TGF-β1-induced VEGF protein level increase in RPE cell supernatants by Elisa.

FIG. 64: Dual luciferase reporter assay showing that miR-15a-5p can target and bind to mRNA of VEGF in vitro. The pmirGLO vector is an empty plasmid, the wild type is a VEGF base sequence, and the mutant type is a VEGF base sequence with different bases in the binding region.

FIG. 65: The intravitreal injection of the Agomir-15a-5p mimic can reverse the increase of hyperoxia-induced VEGF expression levels in the mouse retinas.

FIG. 66: The intravitreal injection of the Agomir-15a-5p mimic can reverse the increase of hyperoxia-induced VEGF expression levels in the mouse retinas.

FIG. 67: The intravitreal injection of the Agomir-15a-5p mimic can reverse the increase of laser-induced VEGF expression levels in the mouse retinas.

FIG. 68: The miR-15a-5p mimic can inhibit the retinal extracellular signal-regulated kinase (ERK) phosphorylation signal activation for a longer time compared to the VEGF monoclonal antibody. FIG. A is a schematic diagram, and the retinas are collected on P13, P14, P15, P17, P20, and P25 after intravitreal injection on postnatal day 12. FIG. B indicates that on P12, the retinal ERK phosphorylation signal of the hyperoxia mouse group is enhanced. FIG. C indicates that on P13, the retinal ERK phosphorylation signal of the VEGF monoclonal antibody group is reduced. FIG. D indicates that on P14, the retinal ERK phosphorylation signals of the miR-15a-5p mimic group and the VEGF monoclonal antibody group are reduced. FIG. E indicates that on P15, the retinal ERK phosphorylation signal of the miR-15a-5p mimic group is reduced. FIG. F indicates that on P17, the retinal ERK phosphorylation signal of the miR-15a-5p mimic group is reduced. FIG. G indicates that on P20, there is no significant difference in the phosphorylation signal of each retinal ERK. FIG. H indicates that on P25, there is no significant difference in the phosphorylation signal of each retinal ERK. FIGS. I, J, K, L, M, N, and O show the statistics of P12, P13, P14, P15, P17, P20, and P25, respectively. FIG. P shows the relative expression level of the retinal phosphorylation ERK signal at different time points of mouse retinas in various groups.

FIG. 69: PCR results showing that miR-15a-5p can reduce Smad2 mRNA level in retinal microvascular endothelial cells.

FIG. 70: Schematic image of western blot results showing that miR-15a-5p can reduce Smad2 protein level in retinal microvascular endothelial cells.

FIG. 71: Statistics of western blot results showing that miR-15a-5p can reduce Smad2 protein level in retinal microvascular endothelial cells.

FIG. 72: Dual luciferase reporter assay showing that miR-15a-5p can target and bind to mRNA of Smad2 in vitro. The pmirGLO vector is an empty plasmid, the wild type is a Smad2 base sequence, and the mutant type is a Smad2 base sequence with different bases in the binding region.

FIG. 73: Immunofluorescent staining results of TGF-1-stimulated retinal microvascular endothelial cell fibrosis markers α-SMA and CD31 after transfection with miR-15a-5p mimic.

FIG. 74: Immunofluorescent staining results of TGF-β1-stimulated retinal microvascular endothelial cell vimentin after transfection with miR-15a-5p mimic.

FIG. 75: Schematic image of change of TGF-β1-stimulated retinal microvascular endothelial cell fibrosis marker vimentin, α-SMA, and CD31 after transfection with miR-15a-5p mimic by western blot.

FIG. 76: Statistical chart of change of TGF-β1-stimulated retinal microvascular endothelial cell fibrosis marker vimentin, α-SMA, and CD31 after transfection with miR-15a-5p mimic by western blot.

FIG. 77: Schematic image of change of TGF-β1-stimulated retinal microvascular endothelial cell phosphorylated-Smad2 and total Smad2 after transfection with miR-15a-5p mimic by western blot.

FIG. 78: Statistical chart of TGF-β1-stimulated retinal microvascular endothelial cell phosphorylated-Smad2 and total Smad2 after transfection with miR-15a-5p mimic by western blot.

FIG. 79: Schematic image of western blot results showing expression levels of fibrosis-associated proteins after stimulation of Müller cells with different concentrations of TGF-β2.

FIG. 80: Statistics of western blot results showing expression levels of fibrosis-associated proteins after stimulation of Müller cells with different concentrations of TGF-β2.

FIG. 81: Schematic diagram of western blot results showing expression levels of fibrosis-associated proteins after stimulation of Müller cells with TGF-β2 following transfection with miR-15a-5p mimic and mimic control.

FIG. 82: Statistics of western blot results showing expression levels of fibrosis-associated proteins after stimulation of Müller cells with TGF-β2 following transfection with miR-15a-5p mimic and mimic control.

FIG. 83: Immunofluorescent staining results of cell marker GS after stimulation of retinal Müller cell with TGF-β2 following transfection with miR-15a-5p mimic.

FIG. 84: Immunofluorescent staining results of fibrosis marker α-SMA and activation marker GFAP after stimulation of retinal Müller cell with TGF-β2 following transfection with miR-15a-5p mimic.

FIG. 85: PCR results showing expression level of TNF-α after stimulation of Müller cells with TGF-β2 following transfection with miR-15a-5p mimic and mimic control.

FIG. 86: PCR results showing expression level of MCP-1 after stimulation of Müller cells with TGF-β2 following transfection with miR-15a-5p mimic and mimic control.

FIG. 87: Schematic image of western blot results showing Smad2 total protein and phosphorylation levels after stimulation of Müller cells with TGF-β2 following transfection with miR-15a-5p mimic and mimic control. P-Smad2 refers to phosphorylated Smad2, and T-Smad2 refers to total Smad2.

FIG. 88: Statistics of western blot results showing Smad2 total protein and phosphorylation levels after stimulation of Müller cells with TGF-β2 following transfection with miR-15a-5p mimic and mimic control. P-Smad2 refers to phosphorylated Smad2, and T-Smad2 refers to total Smad2.

FIG. 89: Cryosections of retinas of hyperoxia-induced mouse groups showing localization of α-SMA and retinal vascular expression.

FIG. 90: Cryosections of retinas of hyperoxia-induced mouse groups showing localization of fibronectin and retinal vascular expression.

FIG. 91: Cryosections of retinas of hyperoxia-induced mouse groups showing localization of α-SMA and retinal activated Müller cell expression. GFAP indicates activated Müller cells.

FIG. 92: Western blot assay showing expression of retinal fibrins. FIG. A shows the expression of retinal fibronectin in hyperoxia-induced mouse groups. FIG. B shows the statistics of fibrin expression level. FIG. C shows the expression of retinal TGFβ receptor 2 protein and α-SMA protein in hyperoxia-induced mouse groups. FIG. D shows the statistics of the expression levels of TGFβ receptor 2 protein and α-SMA protein. FIG. E shows the expression of retinal phosphorylated Smad2 and total Smad2 in hyperoxia-induced mouse groups. FIG. F shows the statistics of the expression levels of retinal phosphorylated Smad2 and total Smad2.

FIG. 93: Safety assessment of intraocular administration of miR-15a-5p for oxygen-induced mouse development. FIG. A shows the body weight change of mice from the day of administration P12 (postnatal day 12) to P42 when the mice are essentially adult (postnatal day 42). FIG. B shows the plasma color of each group. FIG. C shows mouse serum creatinine levels. FIG. D shows mouse serum urea levels. FIG. E shows mouse plasma triglyceride levels. FIG. F shows plasma total cholesterol levels of the mice. FIGs. G and H show H&E staining of the liver and kidney structures of the mice on postnatal day 17 (P17), day 25 (P25), and day 42 (P42).

FIG. 94: Safety assessment of intraocular administration of miR-15a-5p for normal mouse development and retina. FIG. A shows the body weight change of mice from the day of administration P12 (postnatal day 12) to P42 when the mice are essentially adult (postnatal day 42). FIG. B shows mouse serum creatinine levels. FIG. C shows mouse serum urea levels. FIG. D shows mouse plasma triglyceride levels. FIG. E shows plasma total cholesterol levels of the mice. FIG. F shows fluorescent sections of the liver and kidney at different time points after administration. FIGs. G and H show H&E staining of the liver and kidney structures of the mice on postnatal day 17 (P17), day 25 (P25), and day 42 (P42). FIGS. 1, J, and K are OCT results showing the effect of intravitreal injection of the miR-15a-5p mimic, mimic control, and VEGF monoclonal antibody on retinal thickness. FIGS. L and M are cryosections of retinas showing the activation of retinal Müller cells after intravitreal injection of the miR-15a-5p mimic, mimic control, and VEGF monoclonal antibody.

FIG. 95: Therapeutic effect of intravitreal injection of miR-15a-5p mimic on retinal photoreceptor injury and retinal bipolar cell injury of diabetic mouse observed in diabetic mouse (leptin receptor deficiency model mouse). The a wave represents the response of photoreceptor cells to light stimulation, the b wave represents the response of bipolar cells to light stimulation, and the op wave is a set of oscillatory potentials in the b wave and is generally considered to represent the response of bipolar cells-amacrine cells to light stimulation.

FIG. 96: Statistics of therapeutic effect of intravitreal injection of miR-15a-5p mimic on retinal photoreceptor injury of diabetic mouse observed in diabetic mouse (leptin receptor deficiency model mouse). The b wave represents the response of photoreceptor cells to light stimulation.

FIG. 97: Statistics of therapeutic effect of intravitreal injection of miR-15a-5p mimic on retinal bipolar cell injury of diabetic mouse observed in diabetic mouse (leptin receptor deficiency model mouse). The a wave represents the response of bipolar cells to light stimulation.

FIG. 98: Statistics of therapeutic effect of intravitreal injection of miR-15a-5p mimic on retinal bipolar cell injury of diabetic mouse observed in diabetic mouse (leptin receptor deficiency model mouse). The op wave is a set of oscillatory potentials in the b wave and is generally considered to represent the response of bipolar cells-amacrine cells to light stimulation.

FIG. 99: Differences between normal mouse and knockout mouse in superficial retinal vascular plexus growth along astrocyte template on P7. FIG. A shows a representative image of retinal superficial vasculatures with IB4 staining. From left to right, the magnification is 5×, 10×, and 20×. FIG. B shows the statistical results of the area of the retina covered by superficial vasculatures. FIG. C shows the statistical results of the node numbers produced by interlacing of the superficial vasculatures. FIG. D shows the statistical results of the total length of the superficial vasculatures. FIG. E shows the statistical results of the total branch length of the superficial vasculatures. FIG. F shows a representative image of retinal superficial vasculatures with IB4 and GFAP staining. FIG. G shows the statistical results of the node numbers produced by interlacing of superficial vasculatures in the GFAP-positive region. FIG. H shows the statistical results of the total tube length of the GFAP-positive region. FIG. I shows the statistical results of the degree of coincidence of superficial vasculatures and the GFAP-positive region.

FIG. 100: Differences between normal mouse and knockout mouse in superficial retinal vascular plexus and deep vascular plexus growth on P9. FIG. A shows a representative image of retinal superficial vasculatures with IB4 staining. From left to right, the magnification is 5×, 10×, and 20×. FIG. B shows a representative image of a retina covered by retinal deep vasculatures with IB4 staining. FIG. C shows the statistical results of the node numbers produced by interlacing of the superficial vasculatures. FIG. D shows the statistical results of the number of superficial vascular meshes. FIG. E shows the statistical results of the total length of the superficial vasculatures. FIG. F shows the statistical results of the total branch length of the superficial vasculatures. FIG. G shows the statistical results of the coverage area of the deep vasculaturels.

FIG. 101: Therapeutic effect of miR-15a-5p mutant on retinal neovascularization.

### DETAILED DESCRIPTION

The embodiments in the examples of the present disclosure will be described clearly and completely below with reference to the drawings. It is apparent that the described examples are only a part of the examples of the present disclosure, but not all of them. Based on the examples of the present disclosure, all other examples obtained by those of ordinary skills in the art without creative work shall fall within the protection scope of the present disclosure.

The experimental methods involved in the examples are as follows:

1. Effect of miR-15a-5p on Human Retinal Microvascular Endothelial Cells

### 1.1. Cell Culturing

Human retinal microvascular endothelial cells (HRMECs) were purchased from Angiopromie, and cultured in an endothelial cell basal medium (ECM) together with 5% (by volume) fetal bovine serum, 1% (by volume) penicillin-streptomycin, and an endothelial cell growth supplement (ECGS). The cells were incubated at 37° C. with 5% (by volume) carbon dioxide. The culture medium was changed every 2 days. The third to sixth generation cells were taken for study.

1.2. Detection of Cell Proliferation with CCK-8 Kit

A cell counting kit (CCK-8)/WST-8 kit was used: The cells were digested and prepared into a single-cell suspension, and inoculated in a 96-well plate at a density of 2000 cells/well. Five replicate wells were made for each group, and the culture medium was changed after the cells adhered to the wall. A target culture medium was added, and the activity of the cells was detected within 24 h. Before detection, 10 μL of CCK-8 reagent was added to each well, and the mixture was incubated at 37° C. in the dark for 2 h. Subsequently, the incubated culture medium was transferred to an ELISA plate, and the absorbance was measured at 450 nm with a BIO-RAD ELISA reader.

### 1.3. Cell Transfection

HRMECs were inoculated in a 96-well plate at 2000 cells/well and cultured in a cell incubator overnight. 5 μL of Opti-MEM medium was added to two EP tubes, separately. 0.15 μL of Lipofectamine 3000 reagent was added to the first tube, and 0.15 μL of mimic control/mimic/inhibitor control/inhibitor was added to the second tube. The liquids in the two tubes were mixed, and the mixture was incubated at room temperature for 5 min. Subsequently, 10 μL of the mixed solution was added to one well. When preparing a solution, it is generally necessary to calculate the total volume required for all wells. After mixing, the solution is added to each well individually, rather than preparing the solution separately for each well, to avoid errors. Other mimic control, inhibitor control, and inhibitor were transfected into the cells in the same way, but the inhibitor control and inhibitor needed to be added in double the amount of the mimic.

### 1.4. Extraction of Total RNA of Cells

500 μL of Trizol was added to the cells, and the mixture was pipetted and then left to stand at room temperature for 5 min. 100 μL of buffer A was added, and the mixture was shaken vigorously for 15 s and then left to stand at room temperature for 3 min. After liquid separation, the mixture was centrifuged at 4° C. and 15000 g for 5 min. 200 μL of the upper aqueous phase was pipetted into another 1.5 mL EP tube, 1.5-fold volume of absolute ethanol was added, and the tube was turned upside down. The mixture was added into the RNA adsorption column provided by the kit (EZB, Cat. No.: EZB-RN5), and centrifuged at 4000 g for 1 min. The column was taken out, and the liquid in the collection tube was discarded. 500 μL of wash buffer 1 liquid was added to the centrifuge column, and subsequently, the mixture was centrifuged at 12000 g for 1 min. Centrifuging was performed until all the liquid passed through the centrifuge column. Subsequently, 500 μL of wash buffer 2 was added to the centrifuge column, and the mixture was centrifuged at 12000 g for 1 min. The column was taken out, and the liquid in the collection tube was discarded. The mixture was centrifuged at 12000 g for 1 min. The column was taken out, and the liquid in the collection tube was discarded. The adsorption column was transferred to a new 1.5 mL EP tube, and the cap was left open for 2 min. 20 μL of DEPC water was added, and the mixture was centrifuged at 12000 g for 1 min. The centrifuged liquid was added back to the adsorption column, left to stand at room temperature for 5 min, and then centrifuged at 12000 g for 1 min. The liquid in the collection tube was the total RNA, and the concentration was measured with a Nano Drop.

### 1.5. Reverse Transcription

Reagents were added in a 200 μL EP tube according to the following ratios, and the volume of RNA varied depending on the RNA concentration, with a total of 500 ng of RNA. Subsequently, 1 μL of gDNA remover was added, and the mixture was well mixed and stored at room temperature for 5 min. Reagents were added according to Table 1. The sample was placed in a PCR instrument and the PCR instrument was programmed as follows: heating at 37° C. for 15 min, followed by heating at 42° C. for 10 min, and followed by heating at 95° C. for 3 min; and after completion, the obtained cDNA was quickly placed on ice.

TABLE 1

| | |
|---|---|
| 4 × miRNA RT Buffer | 5 μl |
| miRNA RT Enzyme Mix | 2 μl |
| RNA | Proper amount |
| DEPC water | Proper amount |
| Total volume | 20 μl |

1.6. qRT-PCR

A reaction liquid was prepared for each well according to the ratios in Table 2. SYBR Green Master was mixed with cDNA, the upstream and downstream primers were mixed with water, and the insufficient amount of cDNA was made up with RNeasy free water. The mixture was well mixed and then added to a 384-well plate. Care was taken to avoid light. After the sample was loaded, a plate sealer was attached. The sample was centrifuged at 4° C. and 2000 rpm for 3 min, and subsequently, assayed on a machine. The program was set as follows: The first step was 95° C. for 15 min, the second step was 94° C. for 15 s-55° C. for 30 s-70° C. for 30 s and repeated for 40 cycles, and the third step was 95° C. for 15 s-55° C. for 15 s-95° C. for 15 s. U6 was used as an endogenous reference in sEVs study, and cel-miR-39 was used as an exogenous reference in plasma and vitreous body study. The expression level of miRNA was expressed as 2^{\hat{ }Δct}. The primer sequences of each miRNA are shown in Table 3.

TABLE 2

| | |
|---|---|
| SYBR Green Master | 4 μl |
| Upstream primer | 1 μl |
| Downstream primer | 1 μl |
| cDNA | 2 μl |
| DEPC water | 2 μl |

TABLE 3

| | Forward primer | Reverse primer |
|---|---|---|
| U6 | 5′-CTCGCTTCGGCAGCAC | 5′-AACGTTCACGAATTTGCG |
| | A-3′ (SEQ ID NO: 2) | T-3′ (SEQ ID NO: 3) |
| cel- | 5′-ACACTCCAGCTGGGT | 5′- |
| miR- | CACCGGGTGTAAATC-3′ | CTCAACTGGTGTCGTGGAGTCG |
| 39 | (SEQ ID NO: 4) | GCAATTCAGTTGAGCAAGCTG |
| | | A-3′ (SEQ ID NO: 5) |

### 1.7. Cell Scratch Assay

The HRMECs were digested and then inoculated in a 6-well plate, and when the cell fusion degree reached 75%-80%, the cells were transfected with the mimic control, the mimic, the inhibitor control, and the inhibitor, separately. After 6 h, the culture medium containing the transfection reagent was pipetted and removed, and a line was scratched in the center of the cells in each well with the same force by using a 1 mL pipette tip. Subsequently, the cells were washed with a PBS buffer twice to elute the detached cells, and the cells were photographed under a microscope. The photos were saved, and the photographing place was recorded to ensure that the photographing place was the same afterwards. The photographing and saving were performed again after 24 h. The photos were analyzed using image J software to calculate the area of the scratch.

### 1.8. Transwell Cell Migration Assay

The Transwell cell migration assay was used for detecting the effect on cell migration ability after miR-15a-5p transfection. After pretreatment, the HRMECs were trypsinized, and 8000 cells were inoculated in the upper chamber of the well. After incubation for 24 h, the cells were washed with PBS three times. Subsequently, the cells were immobilized with PFA and stained with a 0.01% crystal violet buffer. Under the microscope, 5 fields were randomly selected and the number of cells undergoing migration was counted.

### 1.9. Tube Formation Assay for Endothelial Cells

Matrigel (Corning, Cat. No.: 354234) was thawed at 4° C. for 12 h, and a proper amount of the matrigel was spread on the surface of a 48-well cell plate to support tube formation. Subsequently, the plate was placed at 37° C. for 40 min for curing. After pretreatment, the HRMECs were inoculated in the 48-well plate coated with Matrigel. After 3 h, five areas of each well were collected using an inverted microscope. The total length of the vascular tube and the number of tube nodes were calculated using image-J software.

### 1.10. Immunofluorescent Cell Staining

After pretreatment, the HRMECs were immobilized in 4% paraformaldehyde for 15 min, and subsequently, soaked in PBS with 0.1 Triton-X100 (PBST) for 15 min. After blocking with 5% bovine serum albumin (BSA) at room temperature, the cells were incubated with a primary antibody vimentin (1:300, Abcam, ab45939), a-SMA (1:500, Abcam, ab124964), and CD31 (1:150, Abcam, ab24590) at 4° C. overnight. The cells were washed with PBST three times. Subsequently, the cells were incubated with an Alexa fluor 488-conjugated goat anti-rabbit IgG H&L vimentin (1:1000, Abcam) or an Alexa fluor-647-conjugated goat anti-mouse IgG H&L vimentin (1:1000, Abcam) at room temperature. The nuclei were labeled with 4,6-diamino-2-phenylindole (1:500, Solarbio). The cells were observed under a confocal laser scanning microscope (LSM800, Zeiss Germany) and photographed.

2. Screening and Verification of miR-15a-5p Target Genes

### 2.1. Target Gene Prediction

In three databases (TargetScan, PITA, and microRNAorg), target genes of differentially expressed miRNAs were predicted and screened, separately.

### 2.2. Dual Luciferase Reporter Gene Assay

A luciferase reporter gene assay was used to verify whether Smad2 is a target gene for miR-15a-5p. Dual luciferase reporter plasmids: A pmIRGLO-Smad2 wild type and a pmIRGLO-Smad2 mutant were constructed, with an empty plasmid as a control plasmid. The reporter plasmids were co-transfected into HEK-293 cells with a miR-15a-5p mimic or a scrambled control. 48 h after the transfection, the cells were lysed and the supernatant was collected according to the procedure of a dual luciferase reporter assay system (GenePharma, Cat. No.: G06001). TECAN Infinite 200 (Germany) was used to detect activity of a firefly luciferase reporter gene and a Renilla luciferase reporter gene. For the ratio of the firefly luciferase, the Renilla luciferase was used as relative luciferase activity. Each experiment was repeated 3 times. The process for verifying whether VEGF is a target gene for miR-15a-5p was the same as described above.

### 3. Animal Study Procedures

### 3.1. Establishment of Mouse OIR Model

C57BL/6J mice were used to establish an oxygen-induced neovascular retinopathy model. Neonatal and lactating female mice were exposed to 75% oxygen from postnatal day 7 (P7) to postnatal day 12 (P12) of the neonatal mice. On day 12, the mice were taken out of the oxygen chamber and placed under normoxia. Since hypoxia induces neovascularization, a peak in retinal neovascularization occurred on postnatal day 17 (P17), accompanied by retinal inflammation, nerve injury, and fibrotic changes.

### 3.2. Intravitreal Injection in Mice

On day 12, a modified miR-15a-5p mimic (Agomir) at a concentration of 1 μg was intravitreally injected into mice using a 34-gauge needle (Hamilton, Reno, NV, United States). The same number of mice were injected with a scrambled Agomir as a negative control group. Alternatively, 1 μL of adeno-associated virus was intravitreally injected into the mice at a titer of 1×10¹². After the injection, an antibiotic gel was applied to cover the ocular surface to prevent corneal edema.

3.3. Differences in Absorption of Modified and Unmodified miR-15a-5p by Mouse Retinas

Male 6-week-old C57BL/6J mice were used in this study, and the unmodified miR-15a-5p mimic or the modified miR-15a-5p mimic (Agomir) at a concentration of 1 μg (GenePharma) was intravitreally injected into the mice using a 34-gauge needle (Hamilton, Reno, NV, United States) after anesthesia. The same number of mice were injected with PBS as a negative control group. The retinas were obtained at 8 h, 24 h, 48 h, day 5, and day 7 after the injection for detection of miR-15a-5p levels.

### 3.4 Retinal Flatmounts

The mice were sacrificed on P17, and the eyeballs of the mice were collected. The retinas were detached, fixed in 4% paraformaldehyde, and cut into 4 radial flaps. The retinas were blocked with goat serum for 2 h, and IB4 (Thermo, Cat. No.: 121411) or an anti-glial fibrillary acidic protein (GFAP) antibody (Abcam, Cat. No.: ab7260) was used for retinal vasculature staining. The retinas were washed for 5 h, and then stained with goat anti-rabbit IgG (Abcam 150077) as a secondary antibody. After another 5 h of washing, the retinas were mounted with an anti-fade mounting medium. Retinal vasculature images were captured using a confocal laser scanning microscope (LSM800, Zeiss Germany). Quantitative analysis of neovascularization and non-perfusion regions was performed using Photoshop.

### 3.5. Tissue Immunofluorescence Staining

The eyeballs were dissected and rapidly frozen in an embedding gel. The retinas were sectioned (8 μm) and fixed in 4% paraformaldehyde at room temperature for 20 min. The retina sections were subsequently incubated at 4° C. overnight with a GFAP antibody, α-SMA (Abcam, Cat. No.: ab1224964), Fibronectin (Abcam, Cat. No.: ab45688), and IB4 (Thermo, Cat. No.: 121411), separately. The retina sections were washed with PBS three times, and then incubated with Alexa Fluor 488-conjugated IgG (Abcam, Cat. No.: ab150077) for 2 h. After the retina sections were further washed with PBS three times, the nuclei were labeled with 4,6-diamino-2-phenylindole (1:500, Solarbio). After the retina sections were further washed with PBS three times, the anti-fade mounting medium was dropwise added, and the retina sections were sealed with a coverslip. The retina sections were subsequently photographed with the confocal laser scanning microscope (LSM800, Zeiss Germany).

### 3.6. TUNEL Analysis

The retinal cryosections were subjected to a TUNEL assay using a TUNEL system (Roche, Cat. No.: 11684795910) according to the manufacturer's instructions.

### 3.7. Western Blot Analysis

After BCA quantification, equal amounts of protein samples were mixed with PBS to a final volume of 20 μL, and 5 μL of a protein loading buffer (5×) was added. The mixture was heated at 95° C. for 5 min. The protein electrophoresis conditions were 100 V and 90 min. The electrotransfer conditions were 100 V and 100 min. The electrotransferred PVDF membrane was taken out, blocked, and incubated with a primary antibody overnight. The primary antibody was discarded, and the membrane was washed three times. A secondary antibody was added, and the mixture was incubated on a shaker at room temperature for 2 h. The secondary antibody was discarded, and the membrane was washed three times for 10 min each by adding TBST. An ECL ultra sensitive luminescent liquid reagent was used for developing.

### 3.8. Histopathology and Immunohistochemistry

The eyes were taken out from the mice sacrificed by cervical dislocation on P17. The mice were sacrificed by cervical dislocation on P25 and P42, and the kidneys and livers were obtained and fixed. The specimens were stained using a hematoxylin and eosin (H&E) protocol and imaged using an optical microscope.

### 3.9. Electroretinogram (ERG)

The mice were dark-adapted for 18 h, and the electroretinography procedure was performed according to the instructions (Phoenix Micron VI), with white light flashes emitted in the range of 0.01 to 1 cd-s/m. A notch filter was used to reduce signal noise at 60 Hz, the low-cut filter was set at 0.3 Hz, and the high-cut filter was set at 500 Hz. The amplitudes of the a wave and the b wave were measured. The mice were anesthetized and maintained on a heating pad, and the eyes were moistened with a drop of a balanced saline solution as needed.

### 3.10. OCT

Changes in mouse fundus oculi structures were observed using a SPECTRALIS-OCT (Heidelberg Engineering, Germany). The pupils of both eyes of the mice were dilated with tropicamide eye drops, and after the mice entered the maintenance phase of anesthesia, sodium hyaluronate gel was applied to the eyes of the mice for retinal scanning. The full-thickness of the retinas was scanned with the scanned images centered on the optic disc of the mice.

### 3.11. Establishment of Laser-Induced Mouse Choroidal Neovascularization Model

Male 6-week-old C57BL/6J mice were used in this study, and after anesthesia and mydriasis, laser irradiation was performed on the retina using a Phoenix laser emitter to damage the retinal pigment epithelium layer and the choroid. Neovasculatures from the choroid invaded the retina 7 days later.

### 3.12. Subretinal Injection in Mice

Male 6-week-old C57BL/6J mice were used in this study, and after anesthesia and mydriasis, and a 34-gauge needle (Hamilton, Reno, NV, United States) was inserted through the corneoscleral limbus to lift the retina, and 1 μL of adeno-associated virus was subretinally injected into the mice at a titer of 1×10¹². After the injection, an antibiotic gel was applied to cover the ocular surface to prevent corneal edema.

### 3.13. Fundus Photography and Fundus Fluorescein Angiography

The laser-induced neovascularization model mice, after anesthesia and mydriasis, were used for this study. With the assistance of a transparent gel, the optic disc was centered and photographed using a Phoenix fundus camera. Subsequently, 10% sodium fluorescein (5 mL/kg) was intraperitoneally injected, and after 10 min, fundus photography was performed under a dark field filter to reveal leakage areas.

### 3.14. Quantification of Choroidal Neovascularization

On day 7 after the establishment of the model, the OIR mice were sacrificed by cervical dislocation, and the eyeballs of the mice were collected. The choroids were detached, fixed in 4% paraformaldehyde, and cut into 4 radial flaps. The choroids were blocked with goat serum for 2 h, and IB4 (Thermo, Cat. No.: 121411) was used for choroidal vasculature staining. After 5 h of washing, the choroids were mounted with an anti-fade mounting medium. Choroidal vasculature images were captured using the confocal laser scanning microscope (LSM800, Zeiss Germany). Quantitative analysis of neovascularization was performed using Photoshop.

4. Safety Assessment of Intraocular Injection of miR-15a-5p for Mouse Development

### 4.1. Collection of Blood Samples

Blood samples were collected from the posterior part of the eyeballs and centrifuged at 2000 g for 15 min, and the serum was isolated and subsequently stored at −80° C. The liver, spleen, brain, and kidney were dissected out.

### 4.2. Histopathological Detection

Major organ samples (liver, spleen, and kidney) were fixed in 4% neutral buffered paraformaldehyde for 24 h. paraffin-embedded, sectioned at 3 μm, and stained with hematoxylin and eosin. Micrographs were captured using an Olympus BX51 microscope and an Olympus DP71 CCD camera (Olympus Corporation, Tokyo, Japan).

### 4.3. Determination of Serum Biochemical Parameters

The serum concentrations of creatinine (Elabscience, Cat. No.: EBCK188M), urea nitrogen (Elabscience, Cat. No.: EBCK183M), triglyceride (Elabscience, Cat. No.: EBCK126M), and total cholesterol (Elabscience, Cat. No.: EBCK109S) were determined using a commercially available assay kit. The concentration of each parameter was calculated according to the manufacturer's instructions.

In addition, sources of the reagents used herein are as follows:

The mimic (miR-15a-5p mimic) was purchased from Shanghai GenePharma Co., Ltd., Cat. No.: B02001.

The Agomir (Agomir-15a-5p mimic) was purchased from Shanghai GenePharma Co., Ltd., Cat. No.: B06001: the structure was as follows: The sense strand was SEQ ID NO: 1, and the antisense strand was SEQ ID NO: 6 with two thio-backbone modifications at the 5′ end, four thio-backbone modifications at the 3′ end, a cholesterol modification at the 3′ end, and a full-chain methoxy modification.

The inhibitor (miR-15a-5p inhibitor) was purchased from Shanghai GenePharma Co., Ltd., Cat. No.: B03001.

The Smad2-WT vector was purchased from Shanghai GenePharma Co., Ltd., Cat. No.: C09005-36176.

The Smad2-mut vector was purchased from Shanghai GenePharma Co., Ltd., Cat. No.: C09006-36176.

The VEGF-WT vector was purchased from Shanghai GenePharma Co., Ltd., Cat. No.: C09005-56969.

The VEGF-mut vector was purchased from Shanghai GenePharma Co., Ltd., Cat. No.: C09006-56969.

The adeno-associated virus was purchased from Shanghai GenePharma Co., Ltd., Cat. No.: D08001.

### Example 1: Transfection of Retinal Microvascular Endothelial Cells with miR-15a-5p Mimic and Inhibitor can Increase and Decrease miR-15a-5p Expression Level Respectively

A mimic and an inhibitor of miRNA can simulate the biological effect of miRNA in vitro, the miR-15a-5p mimic is a base sequence of miR-15a-5p synthesized in vitro, and the miR-15a-5p inhibitor is a complementary base sequence of miR-15a-5p synthesized in vitro.

The miR-15a-5p mimic comprises:

a sense strand:
(SEQ ID NO: 1)
UAGCAGCACAUAAUGGUUUGUG;
and
an antisense strand:
(SEQ ID NO: 6)
CACAAACCAUUAUGUGCUGCUA

Firstly, it is necessary to confirm that transfection with the mimic or inhibitor of miR-15a-5p using a lipofectamine 3000 transfection-assisting reagent can indeed increase or decrease miR-15a-5p expression levels in cells. The qRT-PCR results showed that the transfection with the miR-15a-5p mimic can increase the intracellular miR-15a-5p level by about 150-fold (FIG. 1) compared to the mimic control group (scrambled sequence of the mimic), with the differences being statistically significant (P<0.05). The miR-15a-5p level in cells transfected with the miR-15a-5p inhibitor was decreased by 80% (FIG. 1) compared to the inhibitor control group (scrambled sequence of the inhibitor), with the differences being statistically significant (P<0.05).

The retina and choroid contain a large number of vasculatures, of which endothelial cells are important for maintaining vascular permeability. Therefore, selecting human retinal microvascular endothelial cells (HRMECs) for the study is representative. Example 1 indicates that transfecting HRMECs with the mimic and inhibitor of miR-15a-5p in vitro can increase or decrease the expression of miR-15a-5p, and provides a research basis for the subsequent observation of the effect of miR-15a-5p on cells.

### Example 2: Optimal Concentration of VEGF for Stimulating Retinal Microvascular Endothelial Cells to Induce Pathological Proliferation

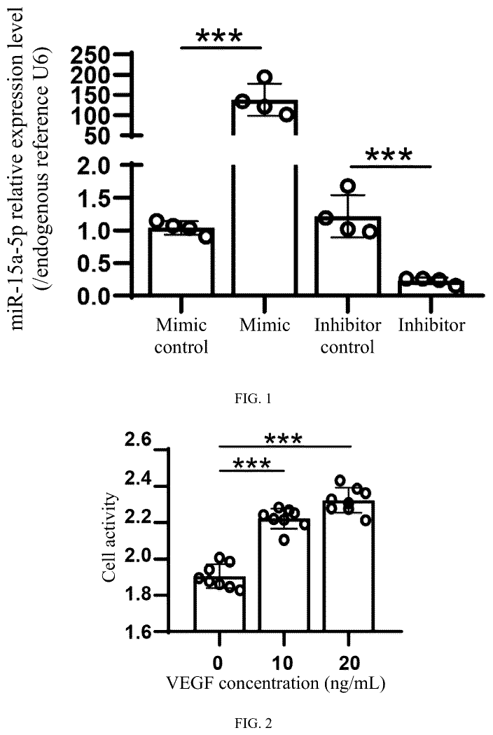

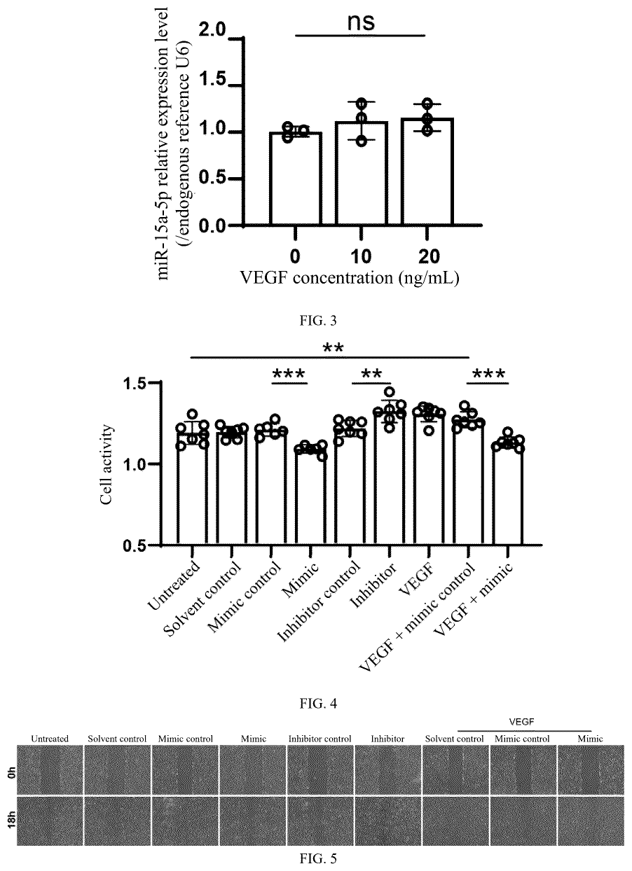

VEGF is a known strong factor inducing in vivo and in vitro angiogenesis, and has strong pathogenic effects in retinopathy of prematurity, diabetic retinopathy, and choroidal neovascularization. In an in vitro experiment, a pathological proliferation model can be established by the action of VEGF on HRMECs. Therefore, the concentration of VEGF for stimulation needs to be determined. The HRMECs were stimulated with VEGF at concentrations of 0 ng/ml, 10 ng/ml, and 20 ng/ml, separately. The CCK-8 assay results showed that VEGF at concentrations of 10 ng/ml or more can significantly induce significant proliferation of the HRMECs (FIG. 2), while VEGF at this concentration did not induce any change in the expression levels of miR-15a-5p in HRMECs (FIG. 3).

### Example 3: Therapeutic Effect of miR-15a-5p on Pathological Proliferation of Retinal Microvascular Endothelial Cells In Vitro

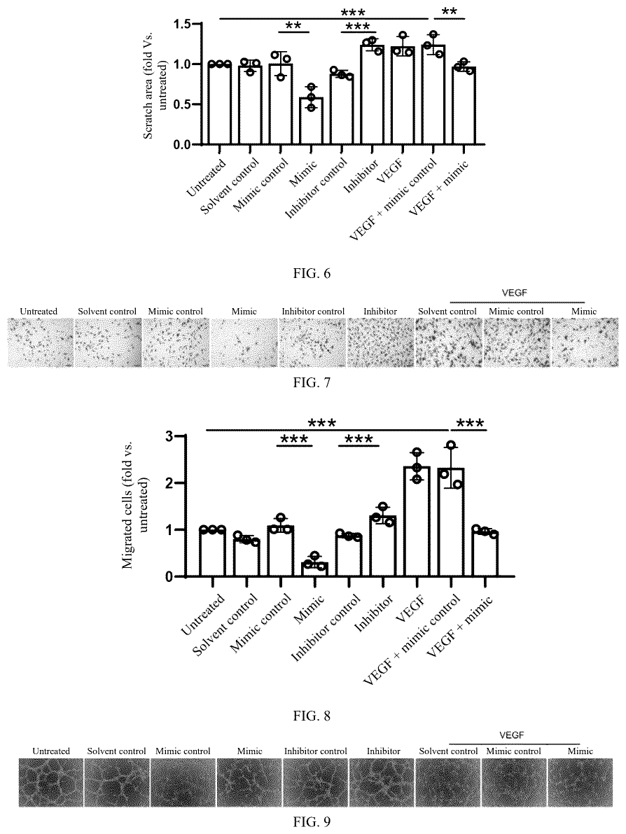

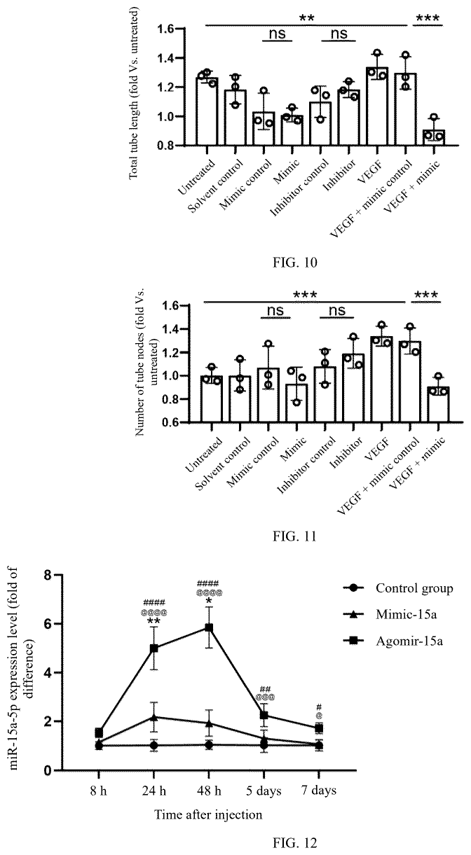

After determining that 10 ng/mL VEGF can induce pathological proliferation of HRMECs with unchanged miR-15a-5p expression levels, the effects of miR-15a-5p on HRMECs under physiological and pathological conditions (10 ng/mL VEGF) were further investigated. The CCK-8 assay is a common method for determining cell proliferation and division activity, and the results showed that the miR-15a-5p mimic can reduce VEGF-induced abnormal cell proliferation (FIG. 4). The cell scratch assay results showed that the miR-15a-5p mimic can attenuate VEGF-induced cell proliferation (FIGS. 5 and 6). The Transwell assay aimed at observing cell migration ability, and the results showed that the miR-15a-5p mimic can attenuate VEGF-induced abnormal cell migration (FIGS. 7 and 8). The tube formation assay simulated the in vitro tube formation ability of the cells, and the results showed that the miR-15a-5p mimic can attenuate VEGF-induced tube formation (FIGS. 9-11). All the differences above are statistically significant (P<0.05).

VEGF can cause endothelial cell proliferation in vivo, which in turn induces the development of neovasculatures. The neovasculatures are different from physiological vasculatures due to the deficiency of tight junctions in neovascular walls. Substances in the neovasculatures leak into the retina and vitreous body through the vasculature walls, resulting in exudation and bleeding in the fundus oculi, which severely affects normal visual function. After the transfection of the HRMECs with the miR-15a-5p mimic in vitro. VEGF-induced abnormal cell proliferation, migration, and tube formation can be reduced. This indicates that the miR-15a-5p mimic has a significant therapeutic effect on the pathological proliferation of HRMECs in vitro.

### Example 4: Enhanced Uptake of Modified miR-15a-5p Mimic by Mouse Retina

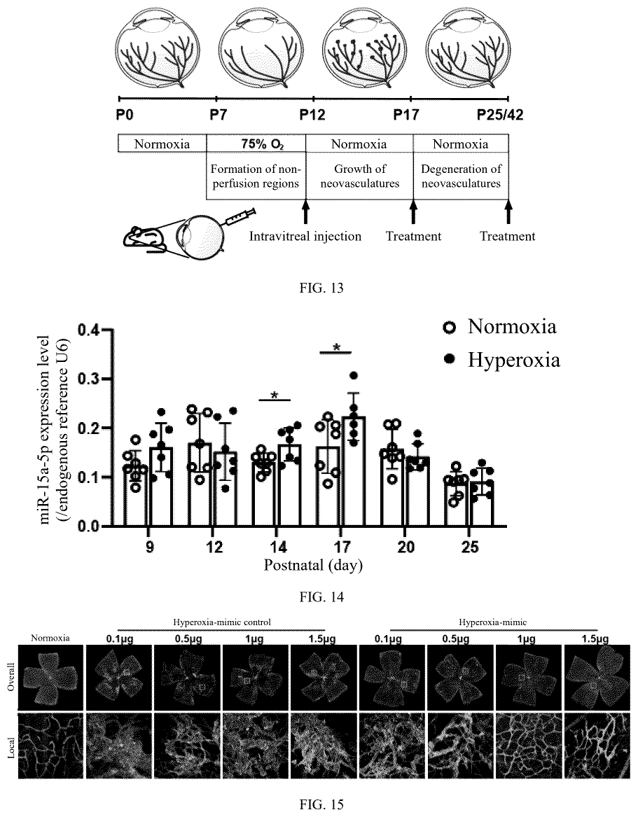

The common mimic of miRNA is called a “mimic”, which is composed of unmodified bases and is well taken up by cells in vitro. In an in vivo experiment, unmodified bases are susceptible to degradation by ubiquitous nucleases in vivo. Therefore, in this example, modified miRNA was used, for example, those with a cholesterol modification and the like. To compare the uptake effect of modified and unmodified mimics by the retina, in this example, the efficiency of mice in the uptake of the same amount of the miR-15a-5p mimic at different time periods was compared. The modified mimic is Agomir. The results, shown in FIG. 12, indicated that significant differences occurred 24 h after the injection of the mimics: the Agomir exhibited good tissue compatibility: the retina showed 2.5-fold greater uptake of Agomir compared to the mimic, and this high level of uptake persisted until day 7 after the injection. Therefore, the Agomir (Agomir-15a-5p) was selected as the miR-15a-5p mimic for in vivo injection in subsequent animal studies.

### Example 5: Therapeutic Effect of miR-15a-5p on Hyperoxia-Induced Mouse Retinal Neovascularization

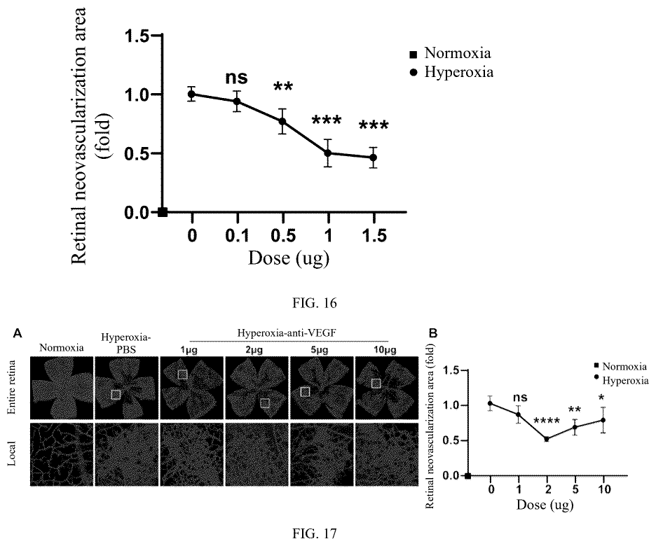

To explore the effect of miR-15a-5p on neovascularization, an oxygen-induced retinopathy (OIR) model was selected. A model construction flowchart is shown in FIG. 13, and this model is the most commonly used retinal neovascularization model and simulates the pathological process of retinal neovascularization (RNV). In this process, specific time points of growth and development were selected to measure the expression levels of miR-15a-5p in the retina. The results, shown in FIG. 14, indicated that the miR-15a-5p expression levels were elevated during the peak period of neovascularization on postnatal days 14 and 17, with the differences being statistically significant. Subsequently, to explore the effect of miR-15a-5p on neovascularization in vivo, a concentration gradient of the Agomir-15a-5p mimic was first set. different doses of the mimic and the mimic control were injected on postnatal day 12. and retinal neovascularization was analyzed by IB4 staining on postnatal day 17. The results showed that the inhibitory effect of Agomir-15a-5p on neovascularization was dose-dependent (FIGS. 15 and 16), and the Agomir-15a-5p mimic at a dose of 0.5-1.5 μg can significantly inhibit the retinal neovascularization (FIGS. 15 and 16).

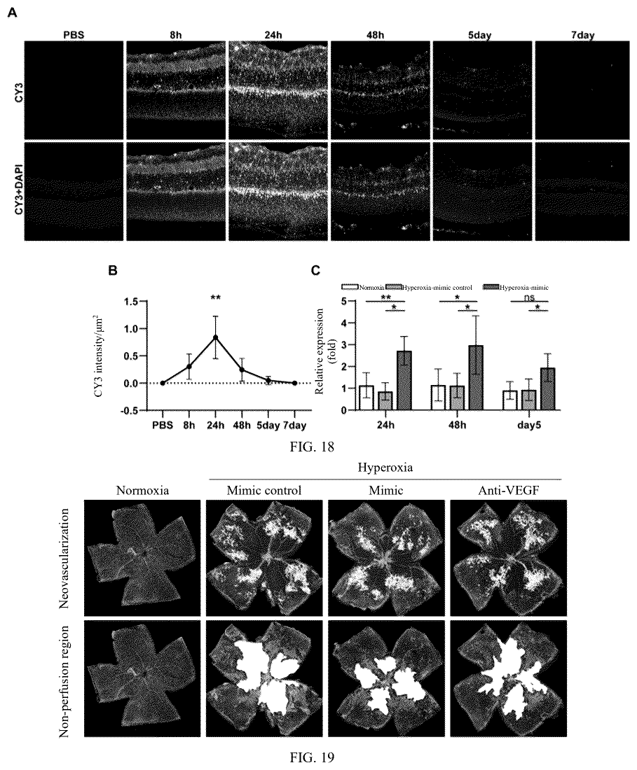

Meanwhile, a VEGF monoclonal antibody was selected as the positive control. The results showed that 2 μg of VEGF monoclonal antibody drug had the optimal inhibitory effect on retinal neovascularization, and subsequently. 2 μg was selected as the therapeutic dose for further verification (FIG. 17). To observe the distribution and duration, of the Agomir in the retina after intravitreal injection. Agomir-15a-5p labeled with CY3 was intravitreally injected into the mice. Agomir-15a-5p labeled with CY3 was distributed diffusely in the retina 8 h after the injection, and the fluorescence intensity peaked 24 h after the injection (FIGs. A and B in FIG. 18). In addition, the fold increase of the Agomir in the retina was detected by the polymerase chain reaction. Agomir-15a-5p increased by about 2- to 3-fold 24 h after the injection and reached a peak concentration of about 3-5 fold 48 h after the injection, with high expression sustained for 5 days (FIG. C in FIG. 18).

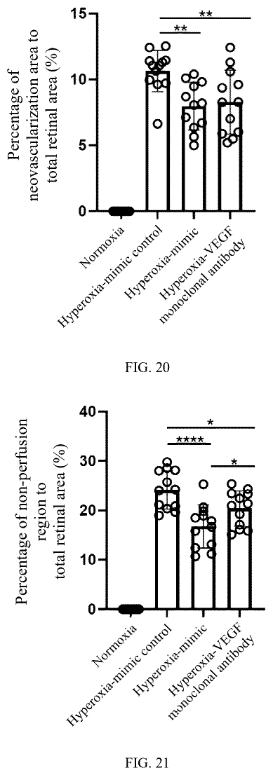

Subsequently, 1 μg was selected as the therapeutic dose for further verification, and meanwhile, the VEGF monoclonal antibody was selected as a positive control. The results showed that the Agomir-15a-5p mimic can reduce retinal neovascularization to 65% of that in the control group (FIGS. 19 and 20). The Agomir-15a-5p mimic can also promote the recovery of the retinal non-perfusion regions compared with the VEGF monoclonal antibody, which reduced the area of the retinal non-perfusion region to 73% of that in the control group (FIGS. 19 and 21).

Adeno-associated virus is a high-efficiency gene delivery tool that can infect retinal cells, inducing high expression of miR-15a-5p. The nucleic acid sequence carried by the adeno-associated virus is TAGCAGCACATAATGGTTTGTG (SEQ ID NO: 9), which can play a therapeutic role after being transcribed into miR-15a-5p in vivo.

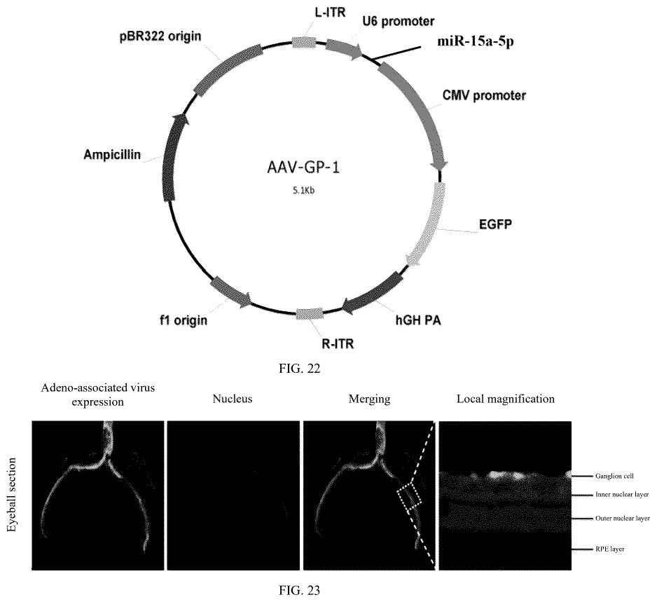

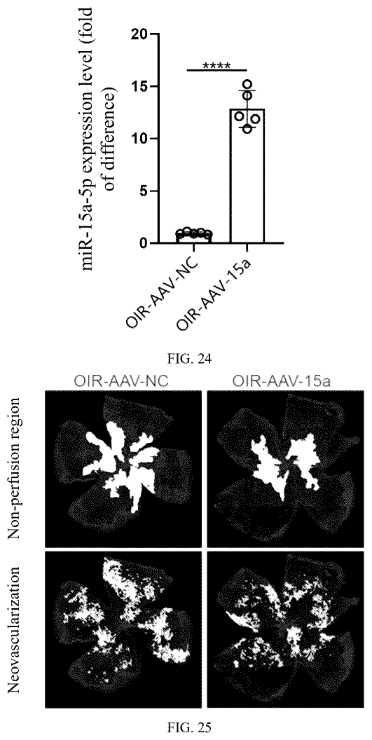

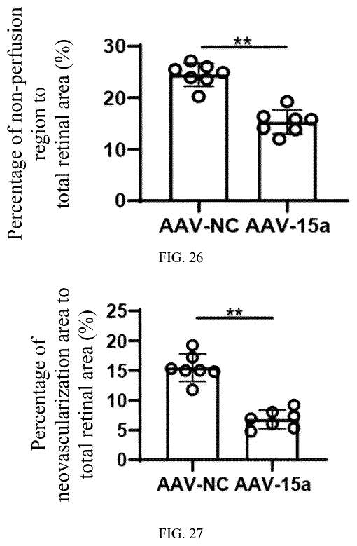

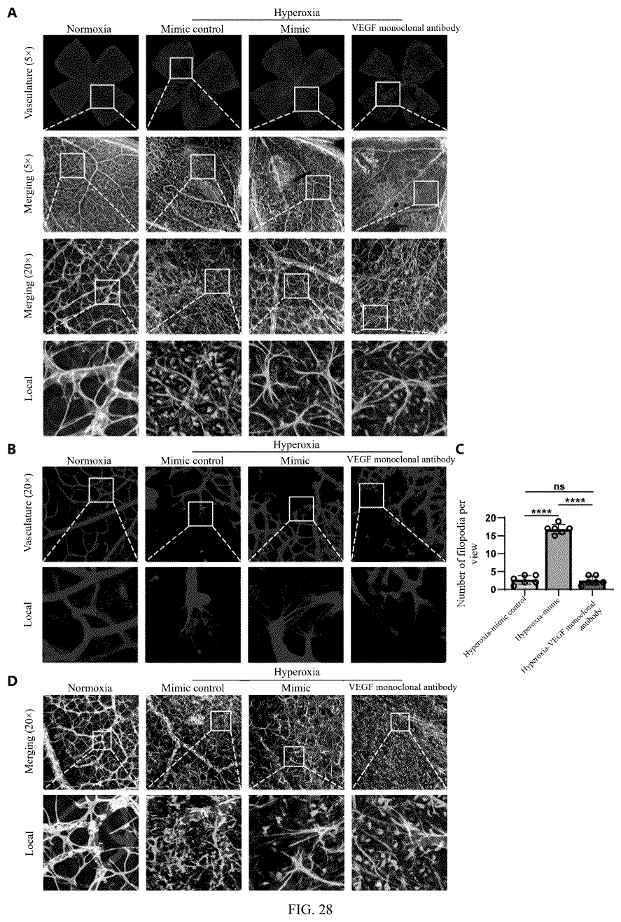

On postnatal day 5 of the oxygen-induced mice, intravitreal injections of the adeno-associated virus comprising miR-15a-5p (1×10¹²), as well as a control virus (1×10¹²) were performed. The virus structure is shown in FIG. 22, the retina was collected on day 12, and the sites of adeno-associated virus infection were observed by eyeball cryosection. The results of FIG. 23 showed that the adeno-associated virus comprising miR-15a-5p infected the retina, with diffuse fluorescence observed in the retina. Subsequently, the retinal miR-15a-5p levels of the groups were detected by PCR, and the results are shown in FIG. 24. The miR-15a-5p level in the retinas of the group injected with the adeno-associated virus comprising miR-15a-5p was 13-fold higher than that of the control group, indicating that the virus can successfully overexpress miR-15a-5p. Retinal neovascularization in the oxygen-induced mice was quantified on postnatal day 17 and the results showed a significant reduction in retinal neovascularization and non-perfusion regions of the group injected with the adeno-associated virus comprising miR-15a-5p (FIGS. 25-27). The results above show that the adeno-associated virus comprising miR-15a-5p can successfully deliver miR-15a-5p to the mouse retinal cells, has a therapeutic effect on hyperoxia-induced mouse retinal neovascularization, and can promote the recovery of the non-perfusion regions.

In Example 5, two methods were used to deliver miR-15a-5p. One was the modified miR-15a-5p, and the other was the adeno-associated virus. The purpose of increasing miR-15a-5p levels in the retina was achieved by intravitreal injection of the two substances. The oxygen-induced retinopathy model used is a classic fundus oculi disease model and can simulate neovascular phenotypes of various fundus oculi diseases, for example, diabetic retinopathy, retinopathy of prematurity, and the like. In this example, the therapeutic effect of miR-15a-5p on neovascularization and non-perfusion regions of this model was observed, and miR-15a-5p was found to have a therapeutic effect on retinal neovascularization and can promote the recovery of the non-perfusion regions.

### Example 6: Promotion of Recovery of Hyperoxia-Induced Mouse Retinal Non-Perfusion Region by miR-15a-5p

The attachment of endothelial tip cells and astrocytes is critical for vascular remodeling in non-perfusion regions. To explain the role of miR-15a-5p in promoting revascularization in non-perfusion regions, we observed the relationship between astrocytes and vascular sprouts in the mouse retinas on day 17. Astrocytes in the vascular occlusive region of the OIR mouse retina degenerated, and the deficiency of astrocytes caused increased GFAP reactivity of the Müller cells, characterized by punctate staining of the Müller cell endfeet in the superficial vascular plexus (FIG. 28A, second column). Specifically, the last row of FIG. 28A shows that in the normoxia group, the retinal astrocytes exhibit a stellate and stretched morphology, while in the hyperoxia control treatment group, the stellate and stretched cellular morphology is absent. Instead, a punctate and irregular cellular morphology is observed, which represents the foot processes of activated Müller cells, indicating increased inflammatory reactivity of the Müller cells. However, compared with the retinas of the untreated OIR group, the astrocytes in the non-perfusion regions of the mouse retina formed a better network and retained their normal stellate/dendritic morphology and density after the Agomir treatment (FIG. 28A, third column). FIG. 28B shows filopodia from the endothelial tip cells of the vasculatures at the edges of the non-perfusion regions, indicating revascularization of the degenerated vasculatures. Our experiments showed that compared with the OIR control group, after the Agomir treatment, the filopodia from the endothelial tip cells near the non-perfusion regions increased by 5-fold (FIG. 28C), and the astrocytes in the non-perfusion regions provided a scaffold and a template for the filopodia to extend from the endothelial tip cells into the occluded retinas (FIG. 28D). Specifically, in the second row of FIG. 28D, in the normoxia group, the vasculatures adhered to the astrocytes without any gaps, while in the untreated hyperoxia group, the vasculatures had no adhesion points for growth, and instead, many punctate cells were observed. After the Agomir treatment, the adhesion of vasculatures to astrocytes was partially recovered, which ultimately promoted the recovery of the non-perfusion regions. The VEGF monoclonal antibody has no reparative effect on the adhesion between the above vasculatures and astrocytes. Therefore, miR-15a-5p-mediated vascular salvage in the retinal non-perfusion regions is associated with the protection of endogenous astrocytes in the vascular occlusion regions.

### Example 7: Effect of miR-15a-5p Deficiency on Hyperoxia-Induced Mouse Retinal Neovascularization and Non-Perfusion Regions

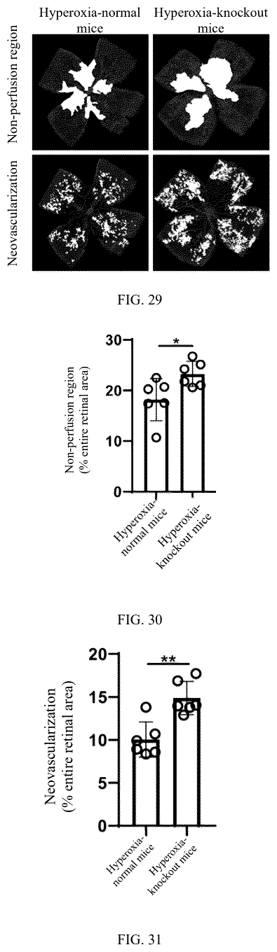

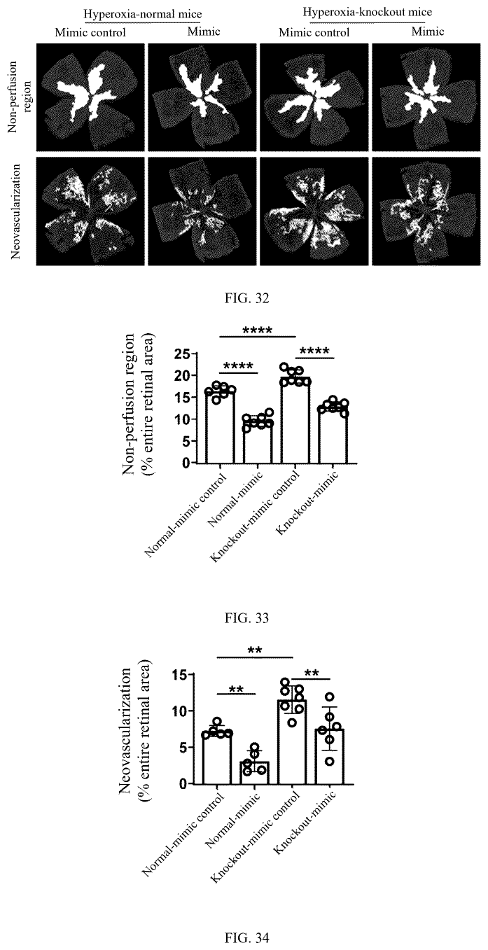

The inhibitory effect of miR-15a-5p on neovascularization was verified in miR-15a-5p knockout mice. The oxygen-induced retinopathy (OIR) model was selected, and the effect of miR-15a-5p in retinal angiogenesis was further verified. The normal and knockout mice were placed in a hyperoxia environment to induce pathological retinal neovascularization, separately (FIG. 29). Areas of retinal neovascular clusters and vascular occlusion were analyzed by IB4 staining on postnatal day 17. The results showed that miR-15a-5p deficiency can significantly increase the areas of non-perfusion regions and neovascularization in the OIR retinas (FIGS. 30 and 31). This indicates that miR-15a-5p deficiency affects angiogenesis in the mouse OIR model. Next, we investigated the effect of the miR-15a-5p mimic (Agomir-15a-5p) on the oxygen-induced neovascularization models of normal and knockout mice to further determine whether miR-15a-5p regulates abnormal retinal angiogenesis (FIG. 32). The results showed that after intravitreal injection of Agomir-15a-5p. not only the areas of the non-perfusion regions and neovascularization in the normal mice retinas can be reduced, but also the increase in areas of the non-perfusion regions and pathological neovascularization in the retinas caused by the miR-15a-5p deficiency in the knockout mice can be reversed (FIGS. 32-34).

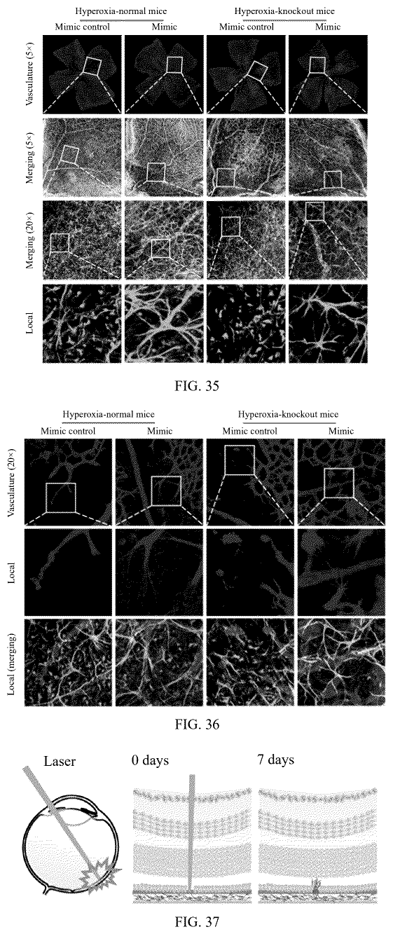

Subsequently, we observed the relationship between astrocytes and vascular sprouts in the retinas of the normal and knockout mice on day 17. Astrocytes in the vascular occlusive region of the OIR mouse retina degenerated. and the deficiency of astrocytes caused increased GFAP reactivity of the Müller cells. In addition, GFAP staining in the knockout mouse retinas was relatively sparse compared with that in the normal mice. The figure showed punctate staining of the Müller cell endfeet in the superficial vascular plexus (FIG. 35). However, in both normal and knockout mice, the astrocytes in the central retina of the Agomir-treated mice formed a better network and retained their normal stellate/dendritic morphology (FIG. 35). Subsequently, we observed filopodia from vascular sprouts at the edges of the non-perfusion regions to observe revascularization of the degenerated vasculatures. The results showed that the Agomir treatment increased the density of the endothelial tip cells in both normal and knockout mouse retinas (FIG. 36), and the astrocytes provided a scaffold and a template for the filopodia to extend from the endothelial tip cells into the occluded retinas (FIG. 36).

### Example 8: Therapeutic Effect of miR-15a-5p on Laser-Induced Mouse Choroidal Neovascularization

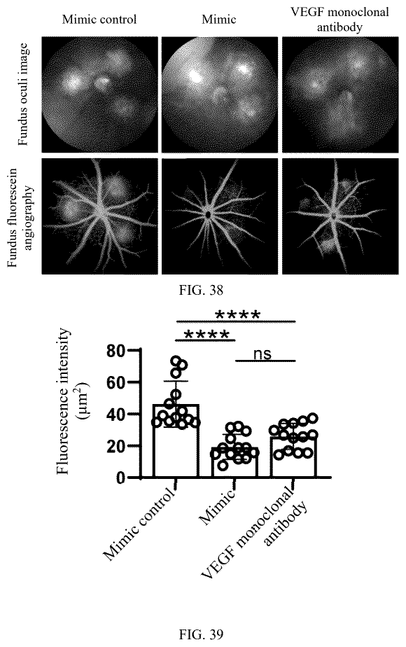

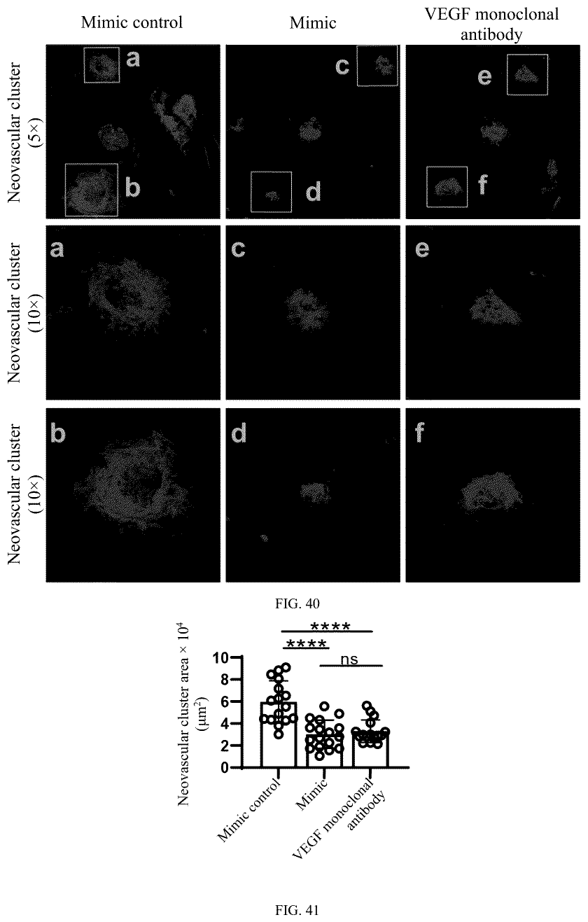

The inhibitory effect of miR-15a-5p on neovascularization was also verified in a choroidal neovascularization model. Firstly, the choroidal neovascularization model was induced by laser, and the model construction process is shown in FIG. 37. In addition, the Agomir-15a-5p mimic, the mimic control, and the VEGF monoclonal antibody were intravitreally injected into the mice. After one week, it was observed by fundus fluorescein angiography that fluorescence leakage areas in both the Agomir-15a-5p mimic group and the VEGF monoclonal antibody group were significantly reduced compared with the control group, indicating that the Agomir-15a-5p mimic and the VEGF monoclonal antibody can inhibit choroidal neovascularization (FIGS. 38 and 39). Choroidal flatmounts from the choroidal neovascularization mice were prepared for IB4 staining to quantify the area of neovascular clusters (FIG. 40), and statistics showed that the formation of choroidal neovascular clusters can be significantly inhibited in both the Agomir-15a-5p mimic group and the VEGF monoclonal antibody group (FIG. 41).

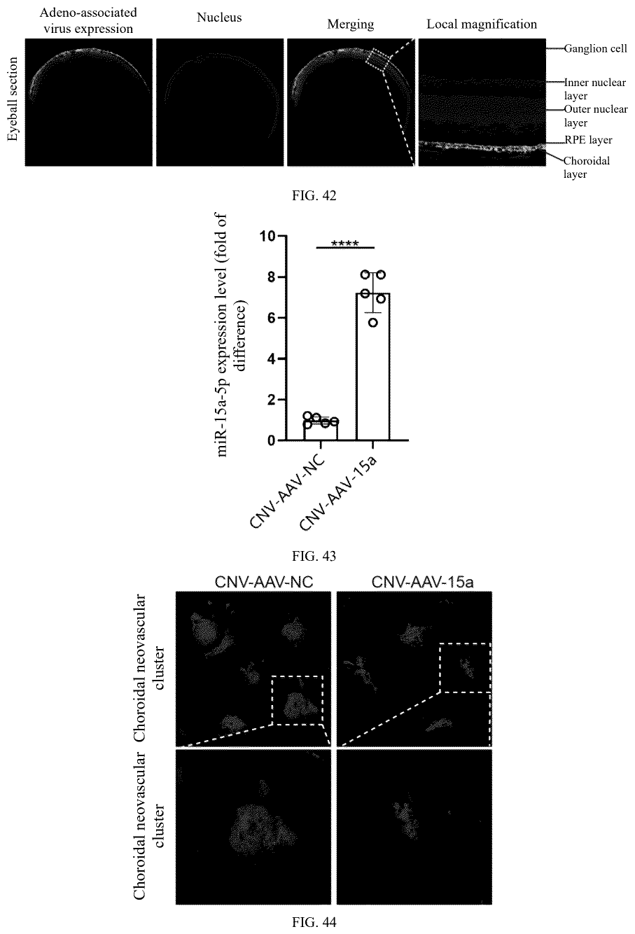

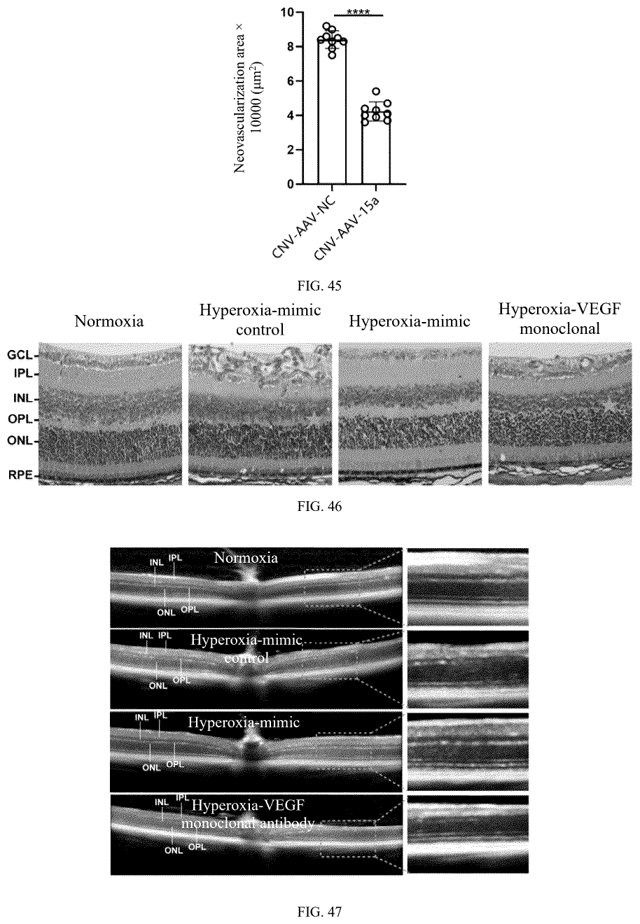

The virus shown in FIG. 22 was also used for the delivery of miR-15a-5p in the choroidal neovascularization model. The nucleic acid sequence carried by the adeno-associated virus is TAGCAGCACATAATGGTTTGTG (SEQ ID NO: 9), which can play a therapeutic role after being transcribed into miR-15a-5p in vivo. 7 days before mouse modeling, subretinal injections of the adeno-associated virus comprising miR-15a-5p (at a dose of 1×10¹²), as well as the control virus (1×10¹²) were performed. On day 7 after the injection, the retina was collected, and the sites of adeno-associated virus infection were observed by eyeball cryosection. The results of FIG. 42 showed that the adeno-associated virus comprising miR-15a-5p infected the retina and choroid, with diffuse fluorescence observed in the retinal pigment epithelium and choroid. Subsequently, retinochoroidal miR-15a-5p levels of the groups were detected by PCR, and the results are shown in FIG. 43. The miR-15a-5p level in the retinas and choroids of the group injected with the adeno-associated virus comprising miR-15a-5p was 7-fold than that of the control group, indicating that the virus can successfully infect the retinas and choroids and overexpress miR-15a-5p. Retinal neovascularization of the mice was quantified on day 7 after the laser inducement and the results showed a significant reduction in retinal neovascularization of the group injected with the adeno-associated virus comprising miR-15a-5p (FIGS. 44 and 45). The results above show that the adeno-associated virus comprising miR-15a-5p can successfully deliver miR-15a-5p to the mouse retinochoroidal cells, and has a therapeutic effect on laser-induced mouse retinal neovascularization.

In Example 8, two methods were also used to deliver miR-15a-5p. One was the modified miR-15a-5p, and the other was the adeno-associated virus. The purpose of increasing miR-15a-5p levels in the retina and choroid was achieved by intraocular injection of the two substances. The laser-induced choroidal neovascularization model used can simulate choroidal neovascularization. In this example, the therapeutic effect of miR-15a-5p on neovascularization of this model was observed, and miR-15a-5p was found to have a therapeutic effect on choroidal neovascularization.

### Example 9: Therapeutic Effect of miR-15a-5p on Retinal Structural and Functional Damage of Oxygen-Induced Mice

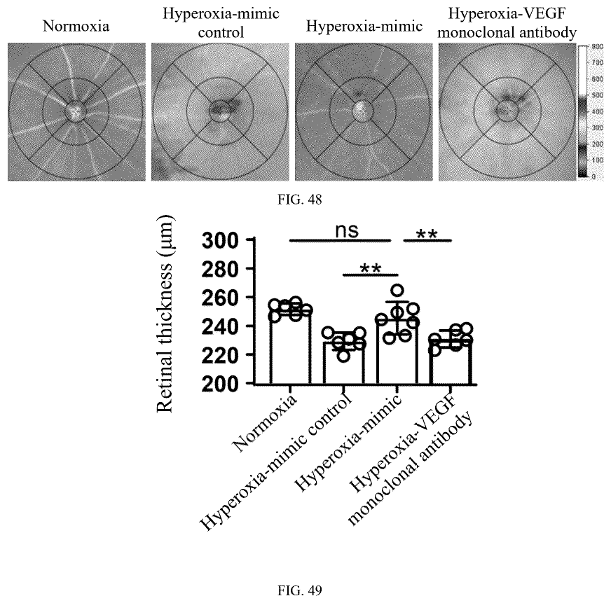

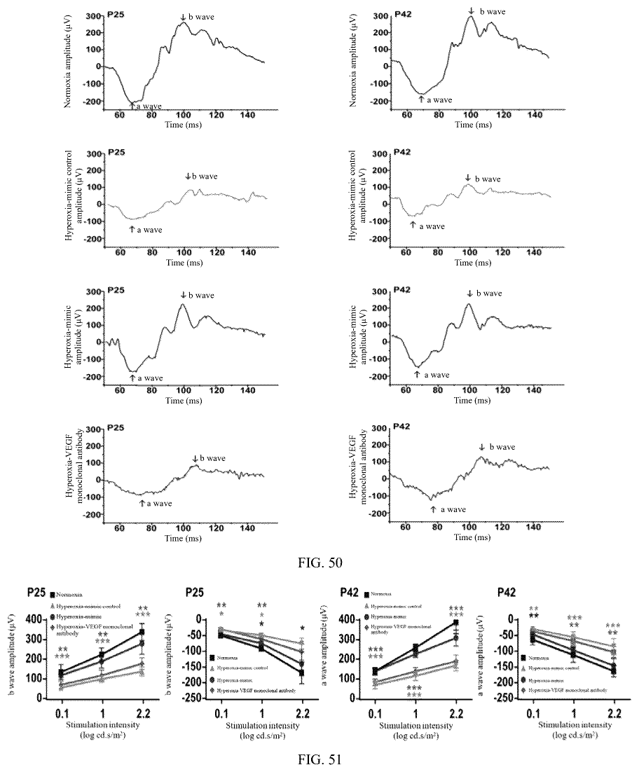

Example 5 shows that miR-15a-5p can recover early blood perfusion in OIR retinas, which is critical for the nourishment and development of retinal nerve cells. In this example, the protective effect of miR-15a-5p on the retina was further assessed in terms of retinal thickness and optic nerve function. Firstly, the structural change of the retina can be directly observed using H&E staining, and FIG. 46 shows that the structure of the mouse retina in the Agomir-15a-5p mimic treatment group was intact, with a normal thickness of the outer plexiform layer (the layer indicated by the star mark), while the mouse outer plexiform layer was significantly thinner in the VEGF monoclonal antibody group. Therefore, the Agomir-15a-5p mimic can significantly ameliorate the retinal structural and functional damage of the oxygen-induced mice. Subsequently, the thickness of each retinal layer was quantified by optical coherence tomography (OCT) (FIGS. 47 and 48), and the results showed that the Agomir-15a-5p mimic can significantly ameliorate the retinal thinning in the oxygen-induced mice, while the retinal thickness in the VEGF monoclonal antibody group showed no significant difference compared with the untreated mice, failing to ameliorate the retinal thinning in the oxygen-induced mice (FIG. 49). Electroretinography (ERG) is widely used for the assessment of retinal function, and the therapeutic effect of the Agomir-15a-5p mimic on oxygen-induced mouse retinal function was observed in early (on postnatal day 25 of the mice) and late (on postnatal day 42 of the mice) stages, separately (FIG. 50). The amplitudes of both the a wave and b wave of the mouse retinas in the oxygen-induced groups were reduced compared with the untreated normoxia group. However, after the Agomir-15a-5p mimic treatment, the amplitudes of both the a wave and b wave of the retinas in the oxygen-induced groups were significantly increased in both the early and late stages. The VEGF monoclonal antibody had no therapeutic effect on retinal neurofunctional damage of the oxygen-induced mice (FIG. 51).

The retina, as a component of the neural tissue, primarily serves to convert light signals into electrical impulses, which are subsequently transmitted to the brain. The structure of the retina is fundamental to its normal visual function, which directly determines the vision of a patient. Various fundus lesions are accompanied by the degeneration of retinal structure and function. In the oxygen-induced retinopathy model used in this example, significant retinal thinning and impaired electrophysiological function were observed after onset. However, after intraocular administration of miR-15a-5p, both retinal structure and neural function showed significant improvement, outperforming the VEGF monoclonal antibody group.

### Example 10: Therapeutic Effect of miR-15a-5p on Chorioretinal Structural and Functional Damage of Laser-Induced Mice

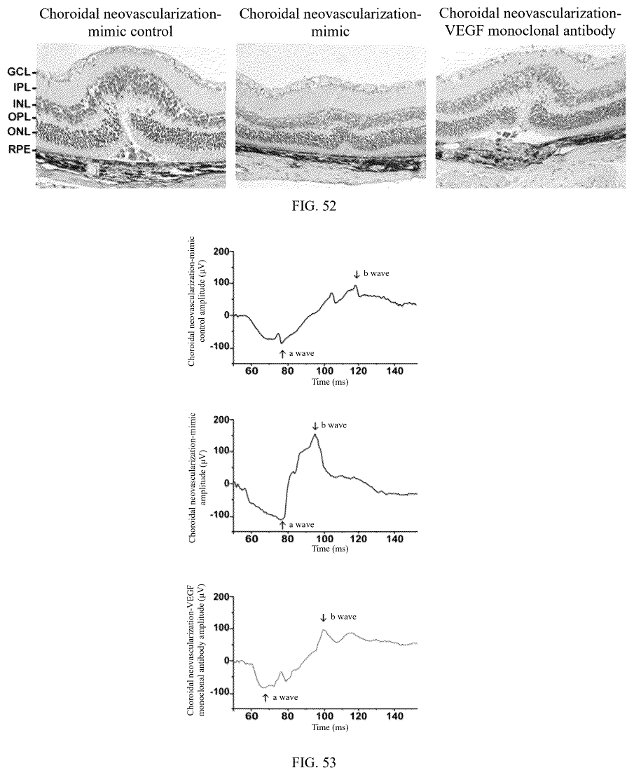

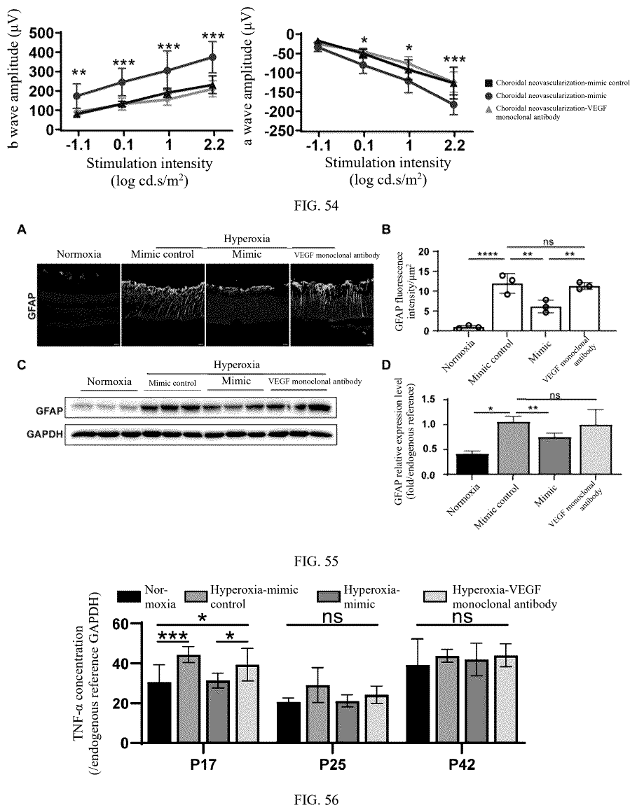

The retinal layered structure, cellular morphology, and lesion of choroidal neovascularization were assessed using H&E staining. The results are shown in FIG. 52. In the control group, neovasculatures from the choroid invaded the subretinal layer, causing a localized elevation in the retina. Fewer elevations were observed in the Agomir-15a-5p mimic treatment group with the lesions substantially resolved, while a few residual lesions remained in the VEGF monoclonal antibody group. The electrophysiological functions of the retinas of the choroidal neovascular mice were assessed using ERG, and the results are shown in FIGS. 53 and 54. The retinal functional damage of the choroidal neovascularization mice in the Agomir-15a-5p mimic treatment group was greatly ameliorated, while no significant treatment effect on the retinal functional damage was found in the VEGF monoclonal antibody group. In addition, under oxidative stress, gliosis occurred in the retina. GFAP is an indicator of glial cell activation, indicating an increase in retinal inflammatory levels. Activation of GFAP in the OIR retinas was significantly reduced after the Agomir-15a-5p treatment (A, B, C, and D in FIG. 55), indicating that miR-15a-5p can inhibit glial cell proliferation in the OIR retinas. However, compared with the control group, the VEGF monoclonal antibody treatment did not demonstrate a tendency to inhibit GFAP activation and was not statistically significant.

In the laser-induced chorioretinal damage model used in this example, significant retinal thinning and impaired electrophysiological function were also observed after onset. However, after intraocular administration of miR-15a-5p, both retinal structure and neural function showed significant improvement, outperforming the VEGF monoclonal antibody group.

### Example 11: Therapeutic Effect of miR-15a-5p on Retinal Inflammation of Hyperoxia-Induced and Laser-Induced Mice

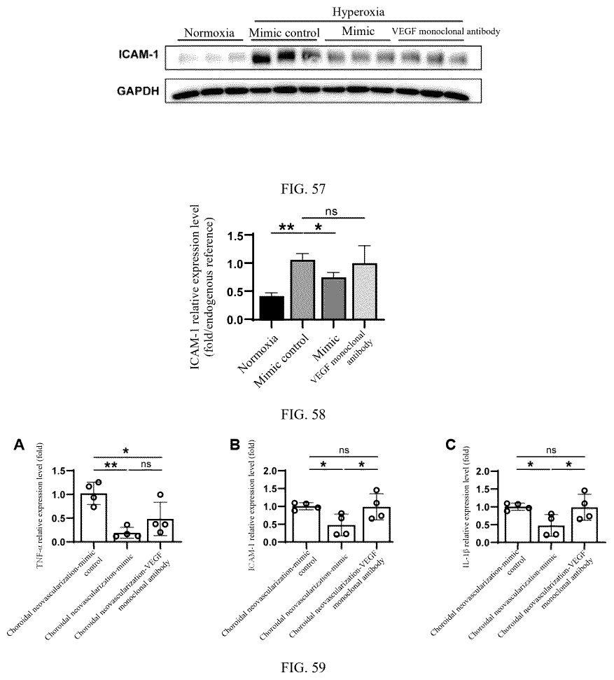

Oxygen-induced mouse retinopathy was accompanied by an increase in inflammatory levels. To explore the therapeutic effect of miR-15a-5p on mouse retinal inflammation, retinas on P17. P25, and P42 were collected to detect TNFα expression levels. The results of FIG. 56 showed that the expression level of the TNFα protein was reduced in the group of mice injected with the Agomir-15a-5p mimic on P17, and no therapeutic effect on the increase of the retinal TNFα level was found in the VEGF monoclonal antibody group. No statistical difference was found in the groups at both time points of P25 and P42. Intercellular adhesion molecule 1 (ICAM-1) is typically expressed in endothelial cells and immune cells and plays a key role in leukocyte migration and activation. ICAM-1 expression levels were increased in the oxygen-induced mouse retinas, and the ICAM-1 expression levels were reduced in the group of mice injected with the Agomir-15a-5p mimic (FIGS. 57 and 58). Choroidal neovascularization occurred in the laser-induced mouse fundus oculi, and this pathology was accompanied by an increase in retinochoroidal inflammation levels. The results of FIG. 59 showed that the injection of the Agomir-15a-5p mimic can reduce the retinal inflammatory factor levels of the laser-induced mice in the groups. The VEGF monoclonal antibody group had no therapeutic effect on retinal inflammation of the laser-induced mice in the groups.

Fundus oculi diseases are often accompanied by an increase in expression levels of inflammatory factors. causing aggravation and prolonged non recovery of retinochoroidal diseases. TNFα and IL-18 are often classical inflammatory factors that recruit inflammatory cells for aggregation. ICAM-1 is an endothelial cell adhesion molecule that can recruit leukocytes to adhere to the surface of endothelial cells, thereby increasing vascular permeability. In this example, the retinal inflammatory factor levels at different treatment nodes were detected, and the results showed that the intraocular administration of miR-15a-5p can significantly reduce the expression level of the retinochoroidal inflammatory factors. The VEGF monoclonal antibody group did not show a significant effect in ameliorating retinal inflammation.

### Example 12: Targeted Regulation of VEGF by miR-15a-5p

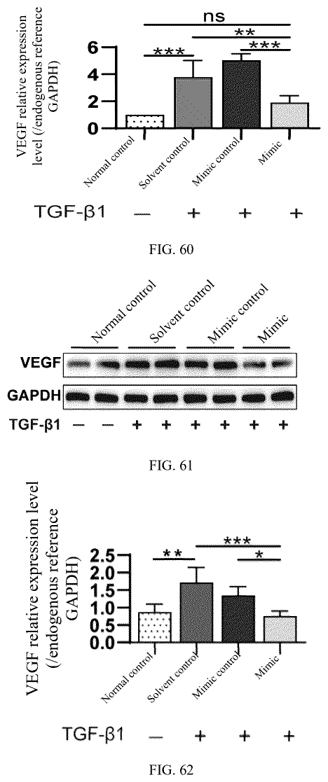

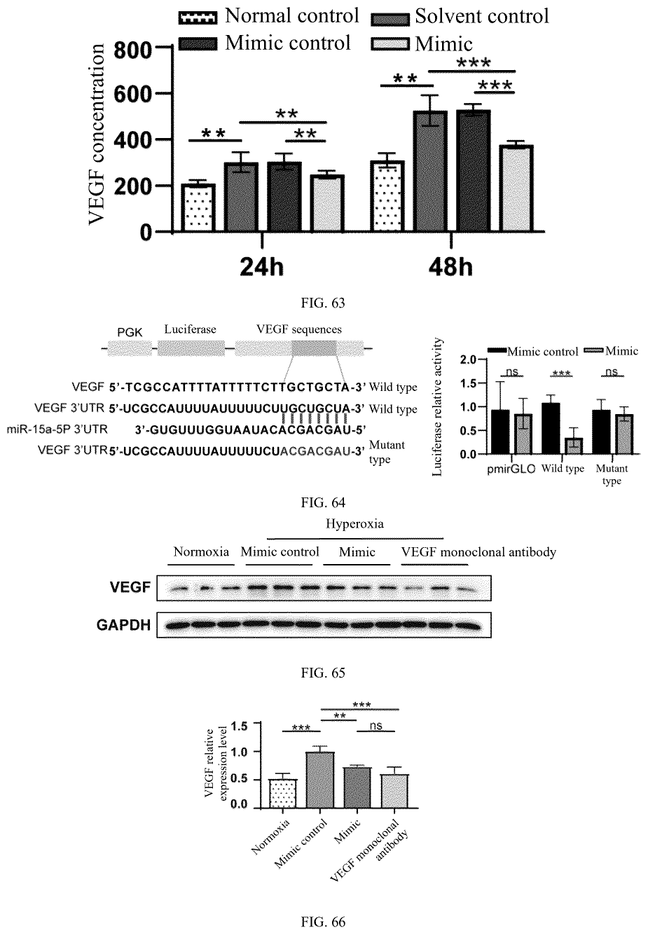

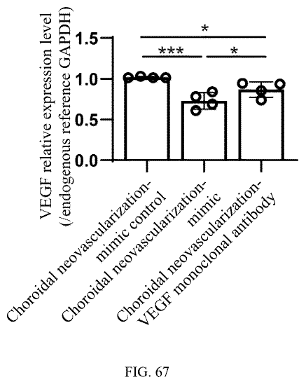

The miR-15a-5p is a base sequence that regulates and controls downstream genes by binding to the RNA of target genes by complementary base pairing, thereby hindering the process of translating the target genes into proteins. In this example, the targeted regulation effect of miR-15a-5p on VEGF was observed through both in vivo and in vitro experiments. The unmodified miR-15a-5p mimic was used in the in vitro experiments, and the modified miR-15a-5p mimic (Agomir) was used in the in vivo experiments. It was found that miR-15a-5p can specifically bind to VEGF mRNA by complementary base pairing, and therefore, subsequent examples were performed in cells and animals for verification, separately. Retinal pigment epithelial (RPE) cells are one of the sources of intraocular VEGF, and it is known that TGF-β1 can induce an increase in VEGF secretion from RPE cells in vitro. Therefore, the miR-15a-5p mimic was transfected into RPE cells, and VEGF secretion was induced by TGF-β1. The results showed that miR-15a-5p can reduce the increase in VEGF expression induced by TGF-1. FIG. 60 showed the mRNA levels of VEGF. FIGS. 61 and 62 showed the VEGF protein levels, and FIG. 63 showed the VEGF protein levels in the supernatant of the RPE cells. FIG. 64 shows the results of the dual luciferase reporter assay. Wild-type VEGF mRNA can bind to miR-15a-5p. while mutant VEGF cannot bind to miR-15a-5p. Therefore, fluorescence quenching only occurred in the group where binding was observed, indicating that miR-15a-5p can specifically regulate and control VEGF transcription. FIGS. 65 and 66 show that the intravitreal injection of the Agomir-15a-5p mimic can reverse the increase of VEGF expression levels in the hyperoxia-induced mouse retinas. FIG. 67 shows that the intravitreal injection of the Agomir-15a-5p mimic can reverse the increase of VEGF mRNA expression levels in the laser-induced mouse retinas.

### Example 13: MiR-15a-5p can Inhibit the Retinal ERK Phosphorylation Signal Activation for a Longer Time Compared with Anti-VEGF

miR-15a-5p specifically binds to VEGF mRNA to inhibit VEGF transcription. The VEGF monoclonal antibody antagonizes VEGF proteins, and the two mechanisms are different. Therefore, when comparing the effects of miR-15a-5p and the VEGF monoclonal antibody, the activation of the downstream VEGF signaling pathway can be selected for observation. After VEGF specifically binds to its receptor VEGFR2, endothelial cell proliferation is activated through signaling pathways such as phosphorylation ERK. Therefore, the ERK phosphorylation signaling pathway was selected as the observation indicator.

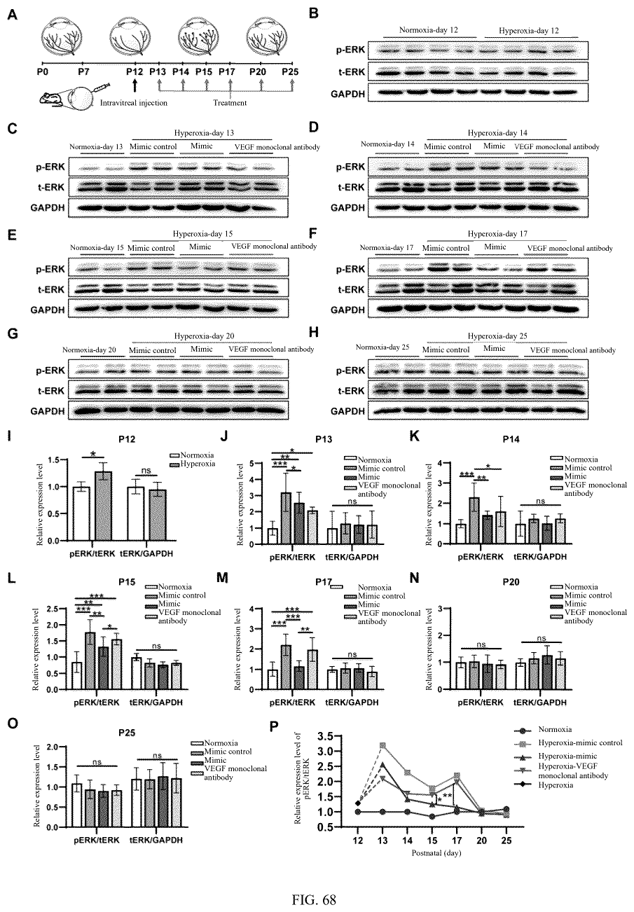

A schematic diagram of the injection and sampling is shown in FIG. A in FIG. 68. The results showed that on the day of the injection, i.e., P12, the phosphorylation ERK signals in the retina in the hyperoxia-induced mouse group were enhanced (FIGs. B and I in FIG. 68): on the first day after the injection, i.e., P13, the phosphorylation ERK signals in the retina in the VEGF monoclonal antibody mouse group first showed a decreasing trend (FIGs. C and J in FIG. 68); on the second day after the injection, i.e., P14, both the VEGF monoclonal antibody group and the Agomir-15a-5p mimic group showed a decreasing trend of phosphorylation ERK signals (FIGs. D) and K in FIG. 68): on the fourth day after the injection, i.e., P15, the ERK phosphorylation signals in the VEGF monoclonal antibody group returned to a high level, while the decreasing trend of phosphorylation ERK signals was maintained in the Agomir-15a-5p mimic group, with statistical differences between the two groups (FIGs. E and L in FIG. 68): during the peak period of neovascularization, i.e., P17 and P15, the decreasing trend of phosphorylation ERK signals was maintained in the Agomir-15a-5p mimic group, and the VEGF monoclonal antibody group showed no inhibitory effect, with statistical differences between the two groups (FIGs. F and M in FIG. 68): during the recovery period of neovascularization, i.e., P20 and P25, no significant difference in phosphorylation ERK signals in the retinas among the groups was found (FIGs. G. H. N, and O in FIG. 68). There was no significant difference in the total protein levels of ERK in the above process, and therefore, a line graph was plotted to illustrate the relative changes in phosphorylation ERK signals for different time periods. As can be clearly seen from FIG. P in FIG. 68, the ERK phosphorylation signals in the retina of the mice in the untreated group increased from P13, peaked on P17, and then gradually decreased, remaining at the highest level throughout. In the VEGF monoclonal antibody group, the inhibitory effect on ERK phosphorylation on P13 was greater than that of the Agomir-15a-5p mimic group. However, starting from P14, the Agomir-15a-5p mimic demonstrated a greater inhibitory effect on ERK phosphorylation than the VEGF monoclonal antibody. A comparison was made between the Agomir-15a-5p mimic group and the VEGF monoclonal antibody group on P15 and P17, and the differences were statistically significant. The differences between the two groups peaked on P17, and on P20 and P25, neither group showed any inhibitory effect on ERK phosphorylation. This indicates that miR-15a-5p can inhibit the ERK signaling pathway more persistently by binding to VEGF mRNA, thereby reducing pathological neovascularization.

### Example 14: Targeted Regulation of Smad2 by miR-15a-5p to Attenuate Retinal Endothelial-Mesenchymal Transition

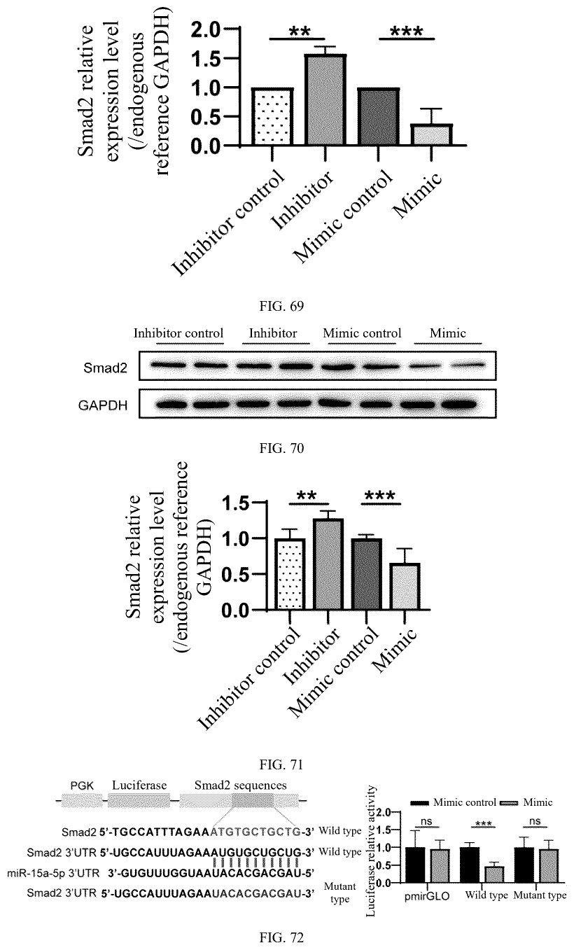

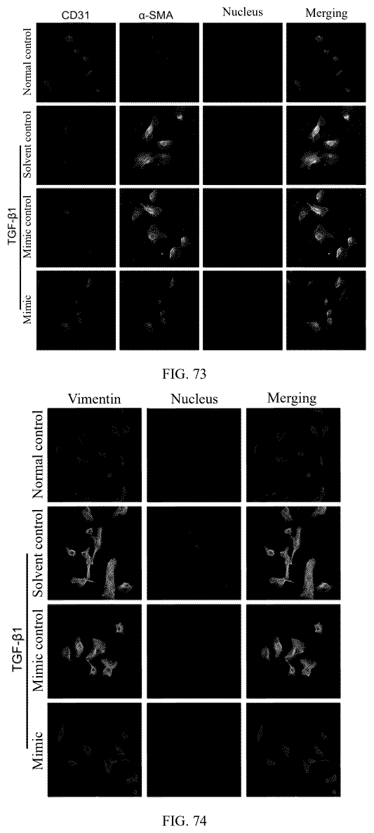

It was found that miR-15a-5p can specifically bind to Smad2 mRNA by complementary base pairing, and therefore, subsequent examples were performed in cells and animals for verification, separately. The unmodified miR-15a-5p mimic was used in the in vitro experiments, and the modified miR-15a-5p mimic (Agomir) was used in the in vivo experiments. Firstly, the mimic and the inhibitor of miR-15a-5p were transfected into the retinal endothelial cells, and PCR (FIG. 69) and western blot (FIGS. 70 and 71) assays showed that miR-15a-5p can inhibit the RNA and protein expression levels of Smad2, while the inhibitor of miR-15a-5p can promote the RNA and protein expression levels of Smad2. This indicates that miR-15a-5p can regulate the expression of Smad2. To determine the direct binding of miR-15a-5p to Smad2, a dual luciferase reporter assay was performed for verification (FIG. 72). The results showed that compared with the control group, the miR-15a-5p mimic significantly reduced luciferase activity of the Smad2 wild-type vector without any effect on luciferase activity of the mutant Smad2. Therefore, miR-15a-5p inhibited the transcription and expression of Smad2 by binding to Smad2 mRNA, i.e., Smad2 was a direct regulation and control target of miR-15a-5p.

Fundus oculi diseases, for example, diabetic retinopathy, neovascular retinal disease, choroidal neovascularization, proliferative vitreoretinopathy, and the like, are accompanied by pathological changes of retinal fibrosis. Smad2, as a part of the TGF-β signaling pathway, is a typical pro-fibrotic pathway protein. Therefore, to verify whether the miR-15a-5p can inhibit the occurrence of retinal fibrosis by inhibiting Smad2. the retinal endothelial cells were stimulated with TGF-1 to induce an endothelial-mesenchymal transition (EndoMT) model to simulate the change of retinal fibrosis, and the miR-15a-5p mimic was transfected into the cells to observe the inhibitory effect of the miR-15a-5p mimic on EndoMT.

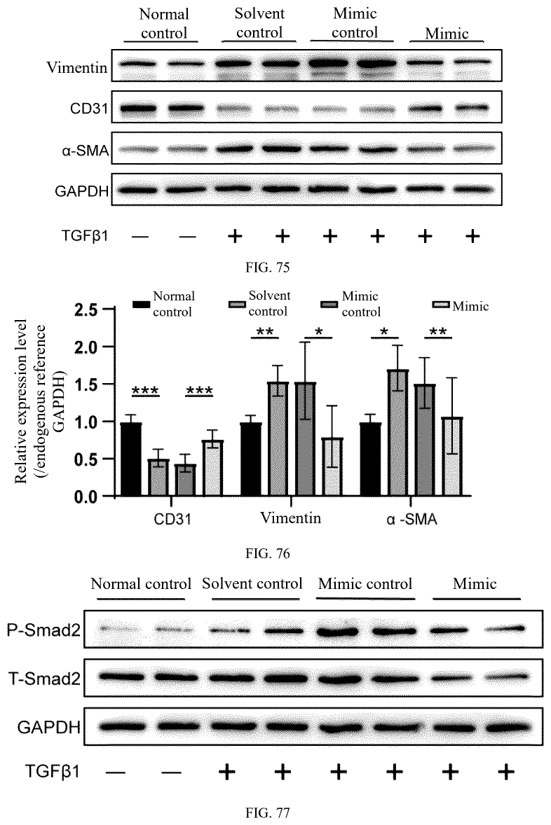

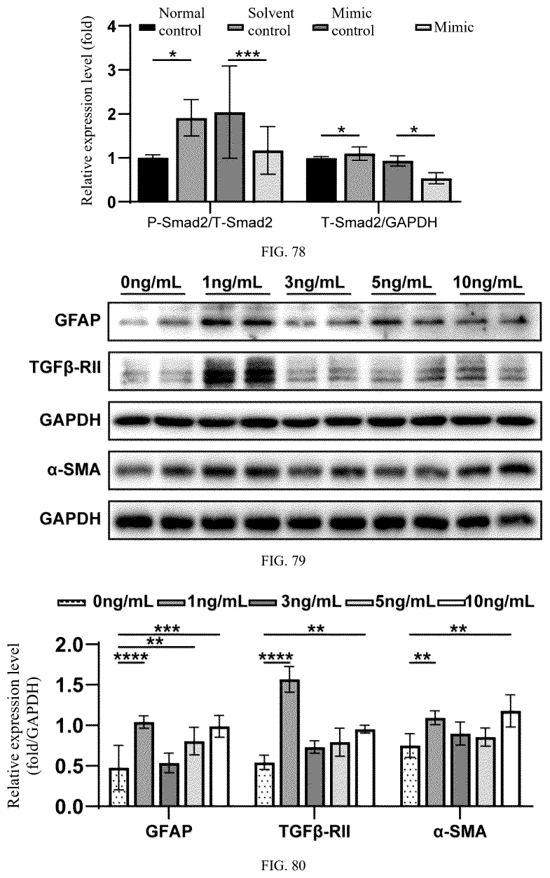

Through immunofluorescence staining (FIGS. 73 and 74) and western blot (FIGS. 75 and 76) assays, it can be directly observed that after stimulation of the endothelial cells with TGF-β1, the expression of fibrotic markers vimentin and α-SMA was significantly increased, while the expression of the endothelial cell marker CD31 was reduced. This indicated that TGF-1 induced the transformation of endothelial cells into fibroblasts, which is known as EndoMT. However, the transfection with the miR-15a-5p mimic reversed the TGF-β1-induced increase in vimentin and α-SMA expression, as well as the reduction in CD31 expression. Thus, miR-15a-5p can reverse TGF-β1-induced EndoMT by inhibiting phosphorylation of Smad2 and expression of total Smad2 (FIGS. 77 and 78).

### Example 15: Targeted Regulation of Smad2 by miR-15a-5p to Attenuate Retinal Müller Cell Fibrosis

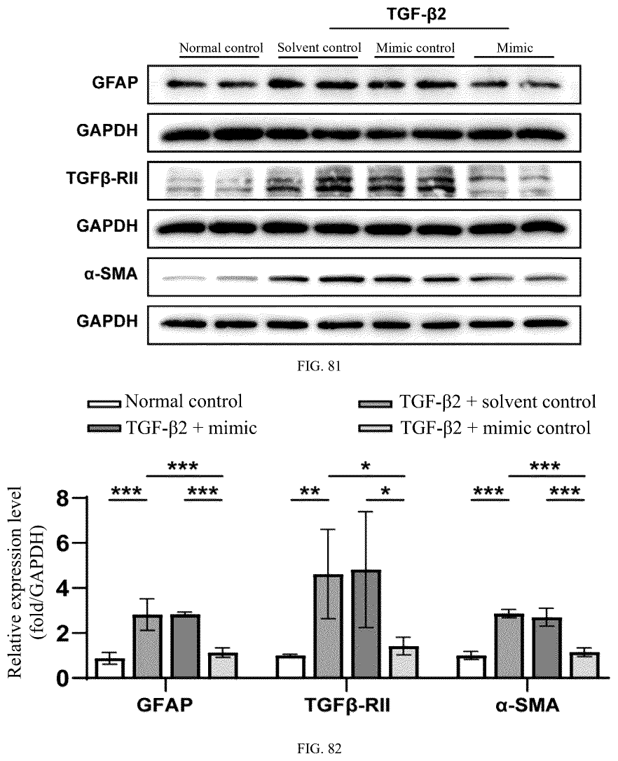

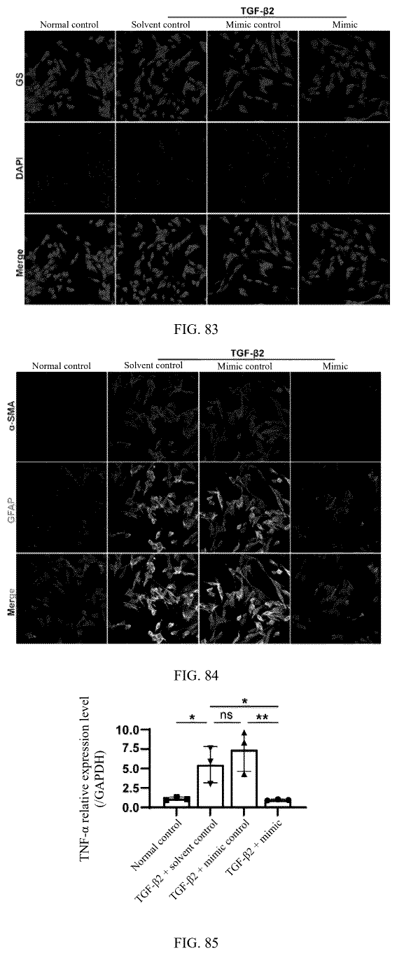

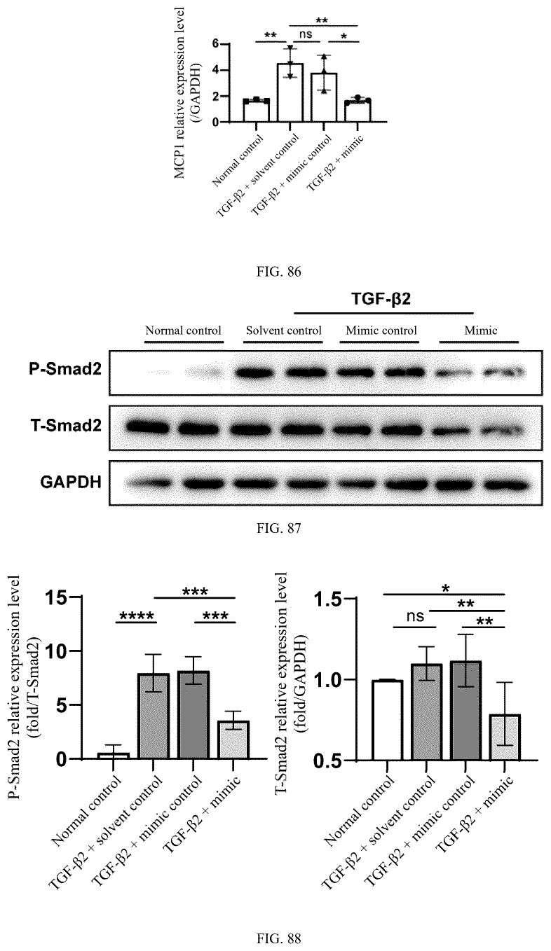

Müller cells are specialized glial cells that span the entire retina, have the function of maintaining the normal structure and function of the retina, and are also involved in various pathological processes, particularly proliferative fundus lesions, such as retinal neovascularization, diabetic retinopathy, and the like. In vitro culture of Müller cells is one way to investigate these proliferative fundus lesions. TGF-β2 can stimulate Müller cells in vitro to develop a fibrotic phenotype. The results of FIGS. 79 and 80 showed that 1 ng/ml TGF-β2 can induce activation of the Müller cells accompanied by an increase in the expression levels of fibrosis-associated proteins. Therefore, in this example, 1 ng/ml TGF-β2 was used for modeling. The Müller cells were transfected with the miR-15a-5p mimic and the mimic control, followed by co-culturing with 1 ng/ml TGF-β2. The results showed that the expression levels of GFAP in the Müller cells transfected with the miR-15a-5p mimic were reduced, and the expression levels of fibrosis-associated proteins were reduced (FIGS. 81 and 82). Further, cellular immunofluorescence staining was performed for observation. Firstly, the Müller cells were identified using the Müller cell marker GS (FIG. 83), and after transfection with the mimic, activation and fibrosis of the Müller cells can be reversed (FIG. 84). In addition, after transfection with the miR-15a-5p mimic, the expression of inflammatory factors including TNF-α and MCP1 in the Müller cells was reduced (FIGS. 85 and 86).

Since it has been confirmed that miR-15a-5p can target and regulate Smad2 to reduce the activation of the TGFβ signaling pathway in retinal endothelial cells, verification was further performed in Müller cells. FIG. 87 showed that the total expression levels of Smad2 in the Müller cells transfected with the miR-15a-5p mimic were reduced, and the phosphorylation signals were weakened (FIG. 88). Therefore, miR-15a-5p can target Smad2 and regulate its expression to attenuate retinal Müller cell fibrosis.

### Example 16: Targeted Regulation of Smad2 by miR-15a-5p to Attenuate Hyperoxia-Induced Mouse Retinal Fibrosis Trend

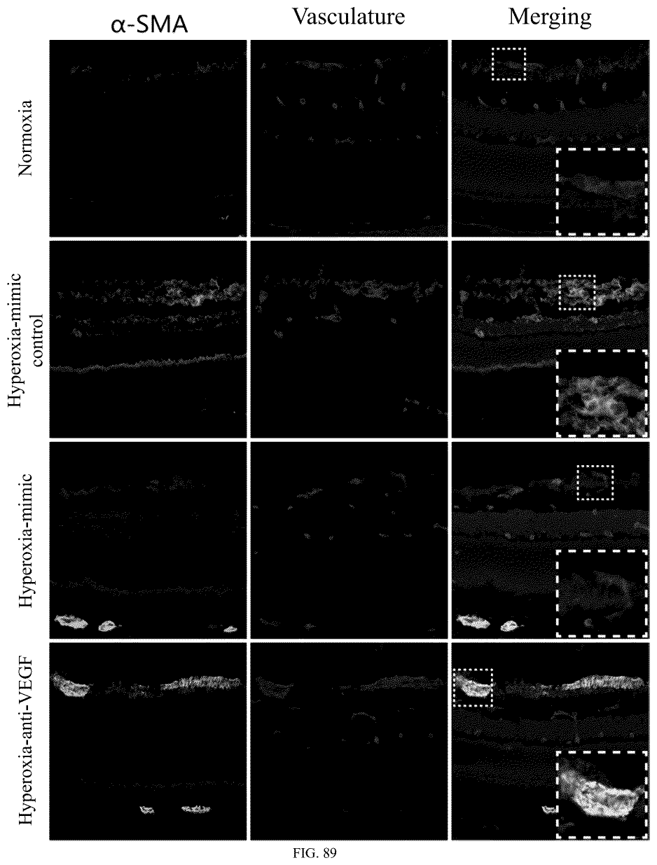

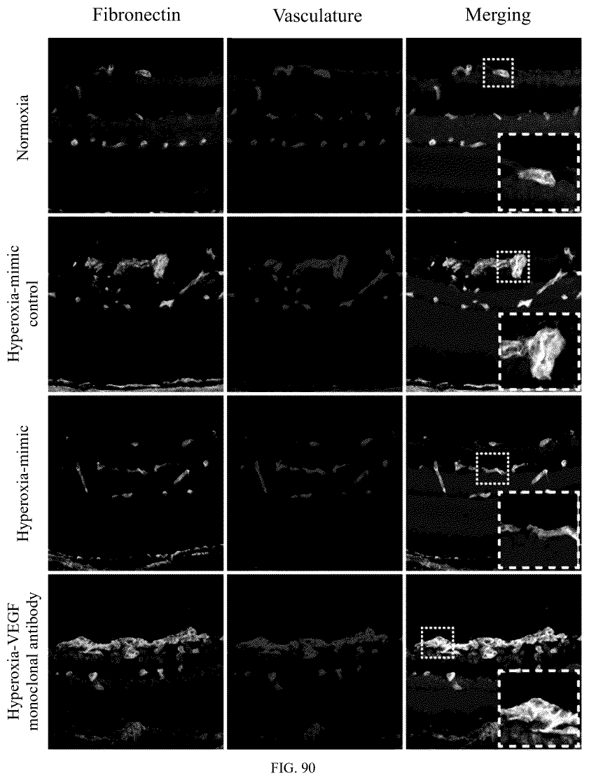

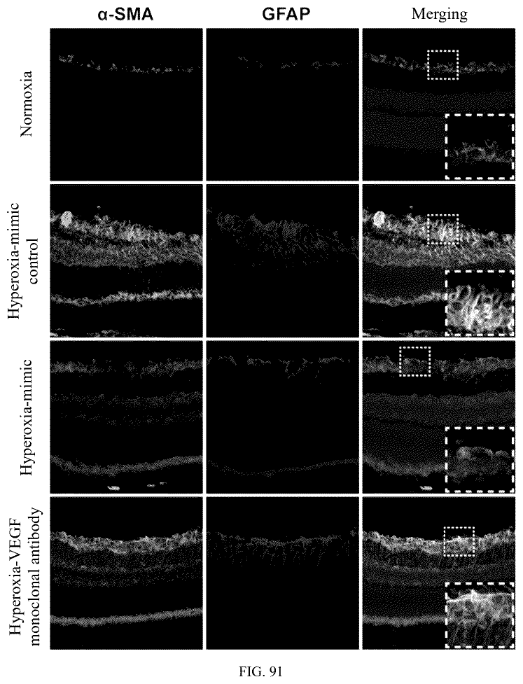

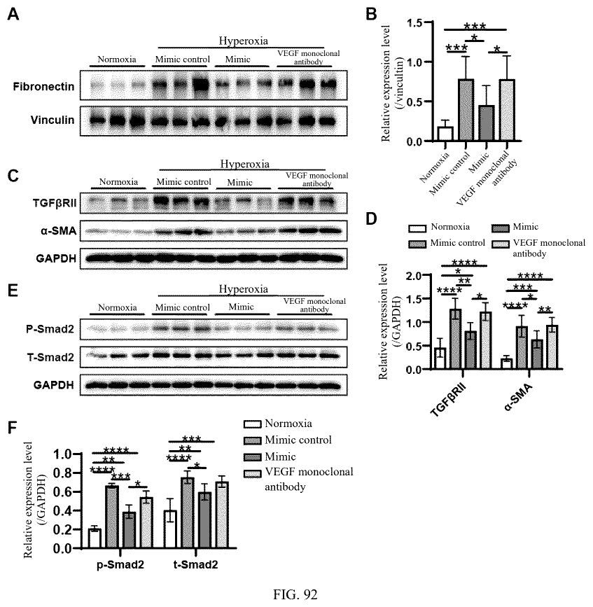

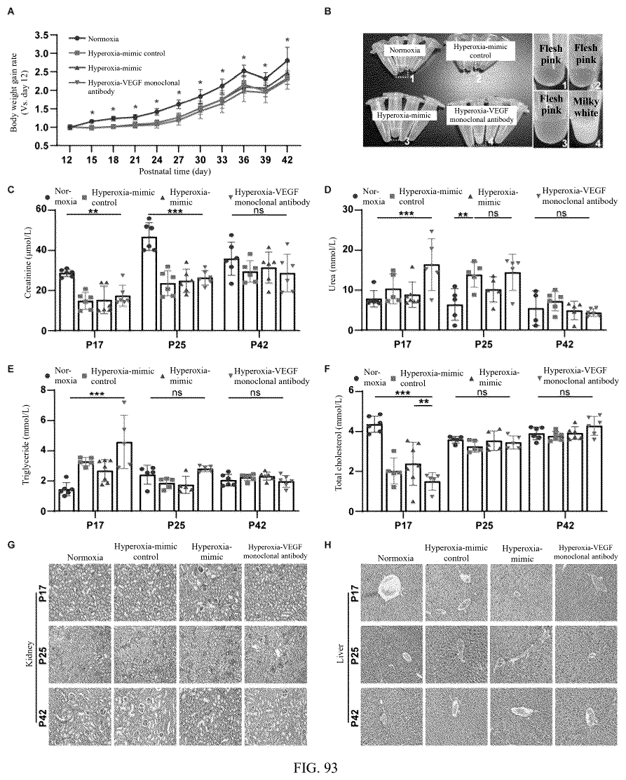

The Agomir-15a-5p mimic, the mimic control, and the VEGF monoclonal antibody were intravitreally injected into the hyperoxia-induced mice. The eyeballs of the mice were cryosectioned on P17, and the proteins of the eyeballs were extracted for western blot assay. FIG. 89 shows co-localization of α-SMA expression and retinal vasculatures in the retina. It can be observed that in the hyperoxia mouse control group, α-SMA had a higher expression level, and showed co-localization with the vasculatures, while in the group with intravitreal injection of the Agomir-15a-5p mimic, α-SMA had a lower expression level, with less co-localization in the retina. FIG. 90 shows localization of fibronectin expression and retinal vasculature. In the hyperoxia mouse control group, the fibronectin had a higher expression level, and showed co-localization with the vasculatures, while in the group with intravitreal injection of the Agomir-15a-5p mimic, the fibronectin had a lower expression level, with less co-localization in the retina, and in the VEGF monoclonal antibody group, the fibronectin had a higher expression level. FIG. 91 shows localization of α-SMA expression and retinal activated Müller cell expression, with GFAP indicating activated Müller cells. The results showed that the co-localization of α-SMA and retinal activated Müller cells was more significant in the hyperoxia mouse control group and the VEGF monoclonal antibody group. This indicates that the Agomir-15a-5p mimic can inhibit the trend of fibrosis in the retina, while the VEGF monoclonal antibody can aggravate the trend of fibrosis in the retina. The results of the western blot assay are shown in FIG. 92, and further confirmed that miR-15a-5p can reduce the expression levels of retinal fibrotic proteins, thereby reducing fibrotic changes in the retina. VEGF monoclonal antibody treatment has no therapeutic effect on retinal fibrosis lesions.

### Example 17: Safety Assessment of miR-15a-5p on Oxygen-Induced Mouse Development

In Examples 5 and 9, the therapeutic effect of intravitreal injection of the Agomir-15a-5p mimic on the neovascularization of the oxygen-induced mice was determined, and then the drug safety of the Agomir-15a-5p mimic was assessed to determine whether the therapeutic regimen has adverse effects on the growth, development, metabolism, and vital organs of the mice. FIG. A in FIG. 93 showed the body weight changes of mice from the day of administration P12 (postnatal day 12) to P42 when the mice were substantially adult (postnatal day 42). The results showed that from postnatal day 15, the body weight gain rate of the mice in the normoxia group was significantly higher than that of the mice in the three hyperoxia groups. However, from the time of administration to adulthood, there was no significant difference in body weight gain rates of the mice in the three hyperoxia groups (FIG. A in FIG. 93). Therefore, intravitreal injection of the Agomir-15a-5p mimic did not adversely affect the growth and development of the mice in the hyperoxia group. To further observe the safety of intravitreal injection of the Agomir-15a-5p mimic, the sera of the mice were collected at three time points of P17, P25, and P42, separately: indicators such as total cholesterol, triglyceride, creatinine, and urea nitrogen were detected; and the liver and kidney functions of the mice in the groups were assessed. As shown in FIG. B in FIG. 93, the plasma color of the mice in the VEGF monoclonal antibody treatment group was different from that of the mice in other groups, indicating that VEGF monoclonal antibody treatment may have an effect on mouse lipid metabolism. As shown in FIG. C in FIG. 93, compared with the normoxia group, the serum creatinine levels of the mice in the three hyperoxia groups were significantly decreased at both time points of P17 and P25, but there was no significant difference among the groups, which may be related to lower body weight. FIG. D in FIG. 93 showed that on P17, compared with other groups, the serum urea levels of the mice in the VEGF monoclonal antibody treatment group were significantly increased. On P25, the serum urea levels of the mice in the hyperoxia control group were significantly increased. There was no significant difference in serum urea levels between the mice in the Agomir-15a-5p mimic treatment group and the normoxia group. FIG. E in FIG. 93 show the plasma triglyceride assay results. Except for P17, the serum triglyceride levels of the mice in the VEGF monoclonal antibody treatment group were significantly increased, and there was no significant difference among other groups. FIG. F in FIG. 93 show the trend of changes of total cholesterol. On P17, the total cholesterol expression levels in all hyperoxia groups were higher than those in the normoxia groups, but there was no significant difference among the hyperoxia groups. In addition, the liver and kidney structures of the mice on P17, P25, and P42 were observed using H&E staining (FIGs. G and H in FIG. 93). The above results indicate that intravitreal injection of the Agomir-15a-5p mimic does not adversely affect the growth, development, metabolism, and organs of the mice in the hyperoxia groups. However, intraocular injection of the VEGF monoclonal antibody can cause abnormal blood lipid metabolism of the mice in the model groups.

### Example 18: Safety Assessment of miR-15a-5p on Normal Mouse Development and Retina

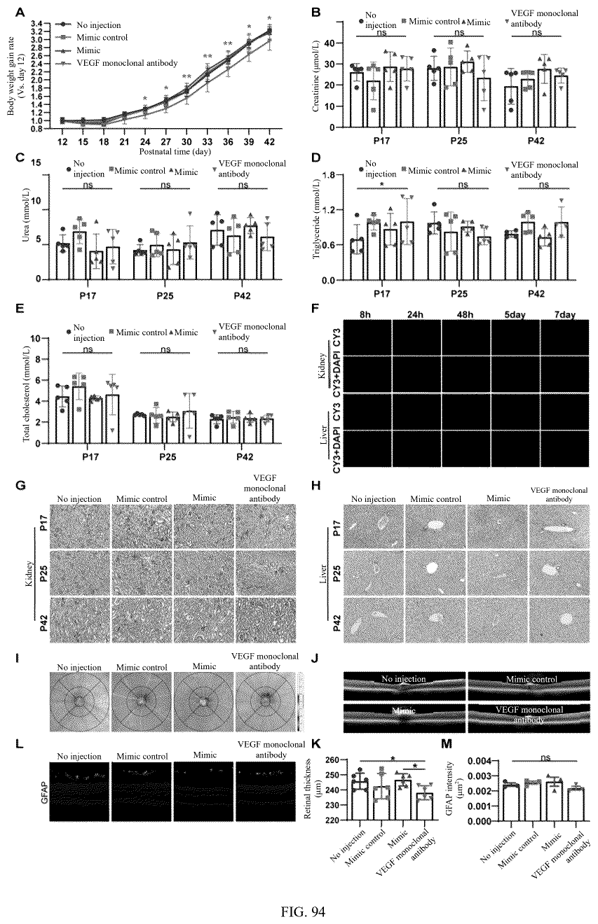

Intravitreal injections were performed on the mice without modeling according to the above therapeutic regimen. The body weights of the mice in the groups were measured, and it was found that starting from P21. the body weight of the VEGF monoclonal antibody group showed a decreasing trend; and on P24, the body weight loss was significant, with statistically significant differences compared with other three groups, which sustained until P42. However, the Agomir-15a-5p mimic did not affect the body weight of the normal mice (FIG. A in FIG. 94). Similarly, mouse sera were collected on P17. P25, and P42 separately to observe indicators such as creatinine (FIG. B in FIG. 94), urea nitrogen (FIG. C in FIG. 94), triglyceride (FIG. D in FIG. 94), and total cholesterol (FIG. E in FIG. 94) to assess the liver and kidney functions related to lipid metabolism of the mice in the groups. It was found that except for the VEGF monoclonal antibody group showing higher triglyceride levels than the normoxia group on P17. no other differences were found, indicating that the VEGF monoclonal antibody affects triglyceride metabolism of the mice. After intravitreal injection of the fluorescent molecule (CY3)-labeled Agomir-15a-5p mimic, the kidneys and livers of the mice were collected for detection. and the results showed no significant fluorescence in the kidneys and livers, indicating that the Agomir-15a-5p mimic did not remain in the kidneys and livers (FIG. F in FIG. 94). H&E staining was performed on the tissue sections of mouse kidneys (FIG. G in FIG. 94) and livers (FIG. H in FIG. 94), and the results showed no significant abnormalities. Intravitreal injections were performed on the mice without modeling to observe whether the intravitreal injection of the Agomir-15a-5p mimic has side effects on the eyes and growth and development of the normal mice. The OCT results showed that the Agomir-15a-5p mimic had no effect on retinal thickness, but the retinas of the mice in the VEGF monoclonal antibody group were thinned (FIGS. I, J, and K in FIG. 94). The GFAP staining showed that intravitreal injection of the Agomir-15a-5p mimic did not cause glial activation (FIGS. 1 and M in FIG. 94). This indicates that intravitreal injection of the VEGF monoclonal antibody can cause delayed development, abnormal lipid metabolism, and thinning of the retina of the mice.

### Example 19: Protective Effect of miR-15a-5p on Diabetic Mouse Retinal Nerve Injury

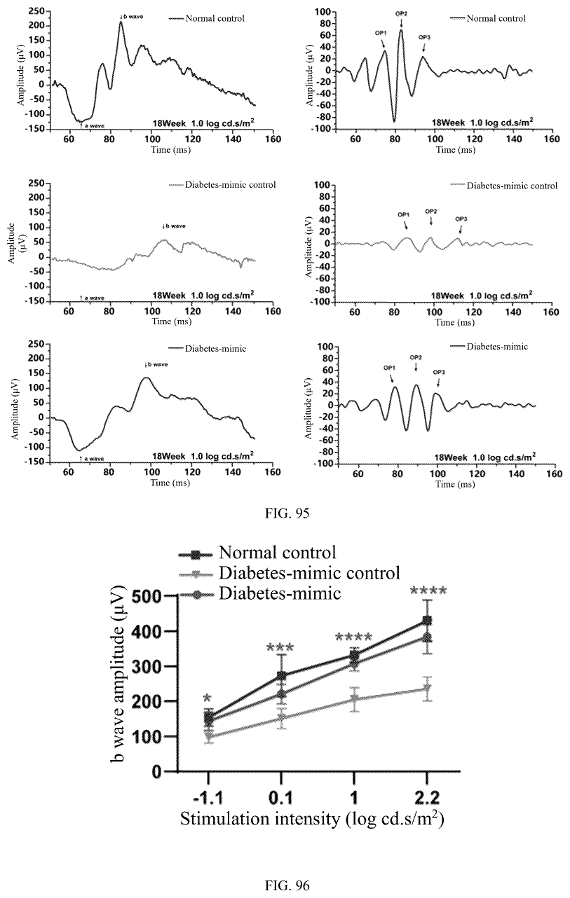

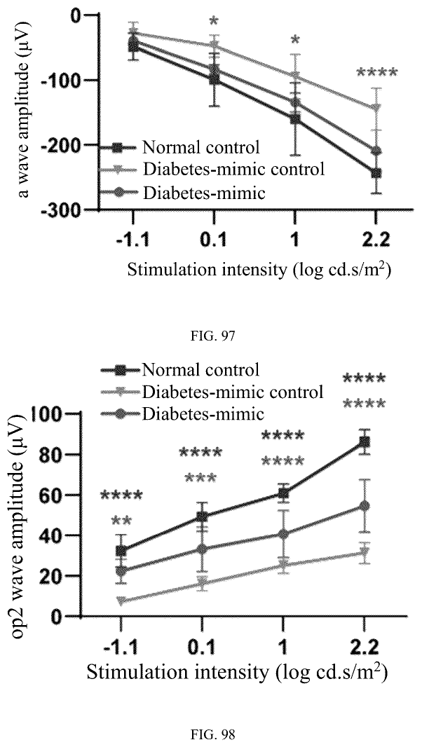

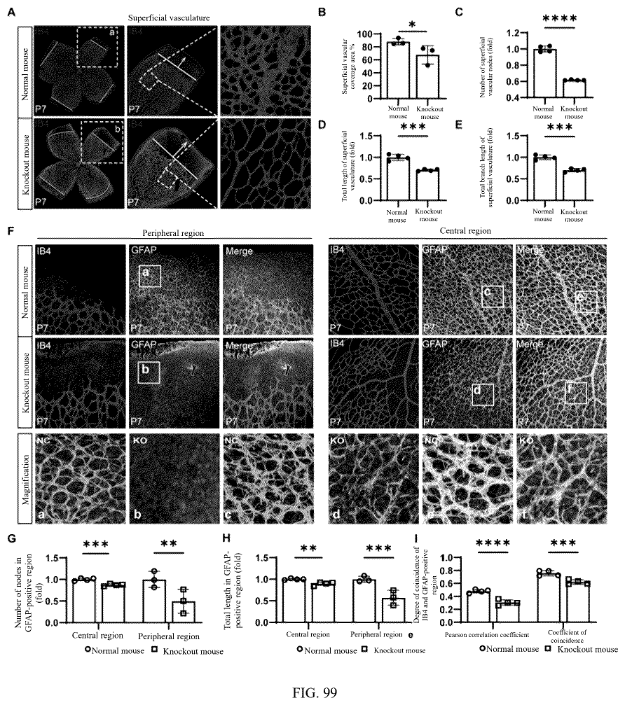

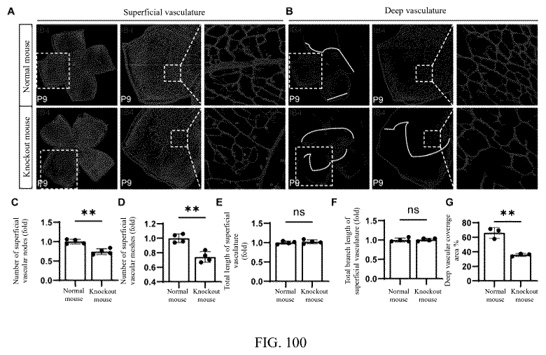

Diabetic patients have high blood glucose levels that can cause irreversible injury to the nervous system. Visual functions of the diabetic patients are significantly reduced. In this example, spontaneous diabetes model mice (leptin knockout mice) were selected as a model of retinal neurodegeneration for study to observe the protective effect of intraocular injection of miR-15a-5p on neurodegeneration. 12-week-old mice were intravitreally injected with the Agomir-15a-5p mimic and the same number of mice were intravitreally injected with the mimic control. The retinal functions of the diabetic mice were detected using electroretinography at week 16. The results showed that the Agomir-15a-5p mimic can significantly improve the amplitude intensity of diabetic mouse retinal photoreceptor cells (FIGS. 95, 96, and 98) and bipolar cells (FIGS. 95 and 97) compared with its control, that is to say, the Agomir-15a-5p mimic has a protective effect on diabetic mouse retinal nerve injury.

### Example 20: Abnormalities in Mouse Vascular Development Caused by miR-15a-5p Deficiency

In mice, the retinal vasculatures originate from the optic disc after birth and spread radially across the inner surface of the retina, gradually forming a superficial vascular network that covers the entire retina. Subsequently, the vasculatures develop towards deeper the layers to form a complete retinal vascular system. During this period, on P7, the superficial vasculatures have already differentiated into main blood vessels and branches, covering 80% of the retinal region and beginning to develop towards the deeper layers. On P9, the superficial vasculatures completely cover the retina, and the deep vasculatures gradually cover 50% of the retina. Meanwhile, astrocytes are closely related to the development of the retinal vascular network. Astrocytes also originate from the optic disc and gradually extend from the disc towards the periphery in the superficial layer of the inner surface of the retina. These cells exhibit a stellate morphology individually and present a reticular structure similar to the vascular network on the whole. The cells develop earlier than the vasculature and provide a template for the development of the retinal vasculature. In this example, we used miR-15a-5p knockout mice to observe the development of retinal vasculatures and astrocytes on P7 and P9. The results of FIG. 99 showed that on postnatal day 7, the development of the superficial vasculatures in the miR-15a-5p knockout mice was delayed, with less retinal coverage compared with the normal mice, with the differences being statistically significant (FIGs. A and B in FIG. 99). This indicates that the deficiency of miR-15a-5p slows down the development of the superficial retinal vasculatures in the mice. Moreover, the total length of the already formed superficial vascular network, the length of the branches, and the number of nodes formed by the crossing vasculatures of the miR-15a-5p knockout mice were all less compared with that of the normal mice (FIGs. C, D, and E in FIG. 99), indicating that the deficiency of miR-15a-5p causes a reduction in the regularity of the vascular network. FIG. F in FIG. 99 shows representative images of co-staining of vasculatures and astrocytes. In normal mice, despite the absence of vascular perfusion in the peripheral regions, a dense reticular structure formed by astrocyte arrangements is still present. However, in the knockout mice, astrocytes are absent in the peripheral regions. Statistically, the number of nodes and the total length of the reticular structure formed by GFAP-positive astrocytes in the knockout mouse retinas were less than those in the normal mice (FIGs. G and H in FIG. 99). The degree of overlap between the vascular network and the reticular structure formed by astrocytes in the knockout mice was lower than that in the normal mice (FIG. 1 in FIG. 99). On postnatal day 9 of the mice, the superficial vascular network substantially covered the retina, but the regularity of the superficial vascular network in the knockout mice was poor (FIG. A in FIG. 100). The number of superficial vascular nodes, the number of superficial vascular meshes, the total length of the superficial vasculatures, and the total branch length of the superficial vasculatures were all lower than those of the normal mice (FIGs. C, D, E, and F in FIG. 100). The development of deep vasculatures is shown in FIG. B in FIG. 100, and the coverage of the deep vascular network in the retinas of the knockout mice was less extensive than that of the normal mice (FIG. G in FIG. 100). Therefore, the absence of miR-15a-5p can cause delayed development of the superficial and deep vasculatures in the retina and abnormalities in the development of astrocytes, which in turn causes the loss of connection between the vascular network and the astrocyte network, that is to say, miR-15a-5p has an important regulation and control effect on the development of vasculatures. In the above 20 examples, Examples 1 and 4 show that the unmodified miR-15a-5p or the modified miR-15a-5p has biological activity, can be absorbed by cells in vitro or by retina cells in vivo, and quickly exerts its biological effect. Examples 2 and 3 show that miR-15a-5p can inhibit pathological neovascularization in vitro. Examples 5-11 show that, in vivo, by means of intraocular administration, miR-15a-5p can inhibit pathological neovascularization, promote recovery of the non-perfusion regions, reduce the expression of retinal inflammatory factors, and promote nerve injury repair. VEGF is a classical pro-angiogenic factor and is involved in the progression of various fundus oculi diseases. Examples 6 and 7 show that miR-15a-5p can rescue astrocytes and provide a template for vascular tip cells, and can significantly promote the recovery of the non-perfusion regions compared with the VEGF monoclonal antibody. The perfusion of vasculatures is crucial for the survival of nerve cells. Example 12 shows that miR-15a-5p can directly target VEGF mRNA and regulate its expression level. Example 13 further compared the duration of the inhibitory effect of miR-15a-5p and the VEGF monoclonal antibody on the retinal neovascularization signaling pathway, and the results show that miR-15a-5p can exert a more sustained inhibitory effect. Fundus oculi diseases, for example, diabetic retinopathy, neovascular retinal disease, choroidal neovascularization, proliferative vitreoretinopathy, and the like, are accompanied by pathological changes of retinal fibrosis. Smad2, as a part of the TGF-β signaling pathway, is a typical pro-fibrotic pathway protein. Examples 14-16 show that miR-15a-5p inhibits the onset of retinal fibrosis by inhibiting Smad2. Examples 17 and 18 assessed the effects of intraocular injection of miR-15a-5p and the VEGF monoclonal antibody on oxygen-induced mouse development and normal mouse development. The results show that the VEGF monoclonal antibody can cause body weight loss and dyslipidemia of the normal mice, while miR-15a-5p has no adverse effect on mouse development. Example 19 shows that miR-15a-5p can alleviate retinal neurodegeneration of the diabetic mice. Example 20 shows that the absence of miR-15a-5p can cause delayed development of the superficial and deep vasculatures in the mouse retina and abnormalities in the development of astrocytes, which in turn causes the loss of connection between the vascular network and the astrocyte network, and affects retinal development, that is to say, miR-15a-5p has a crucial regulation and control effect on vascular development. In conclusion, miR-15a-5p or modified miR-15a-5p demonstrates a clear therapeutic effect in treating fundus oculi diseases, offering substantial application and research values in the field of biomedicine.

### Example 21: Therapeutic Effect of miR-15a-5p Mutant on Retinal Neovascularization

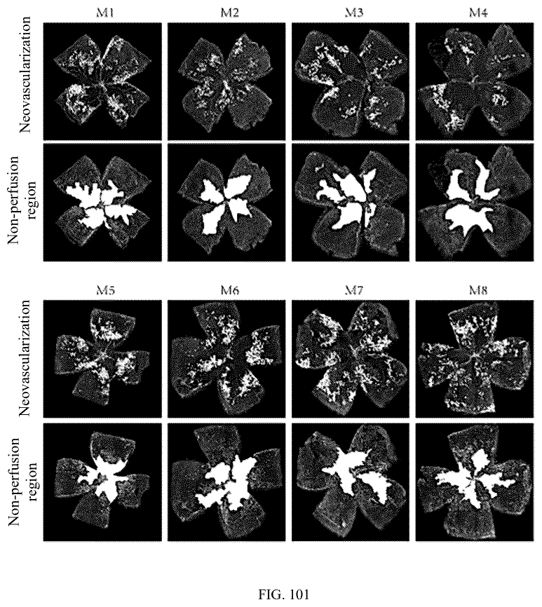

Examples 1-20 have confirmed that miR-15a-5p or the modified miR-15a-5p demonstrates a clear therapeutic effect in treating fundus oculi diseases. In this example, mutations were further introduced on the basis of miR-15a-5p to determine the effect of a miR-15a-5p mutant in treating fundus oculi diseases. The mutation sites are shown in Table 4, where the bold sequences represent seed sequences (sequences that must be included for treating diseases), and the underlined sequences represent mutated sequences, with mutations at 1, 2, 3, and 5 sites, separately. Subsequently, the mutated sequences were used to treat the retinal neovascularization model, and the results are shown in Table 4 and FIG. 101. The wild-type sequence can inhibit neovascularization by up to 74.8%, and non-seed mutated sequences can still exhibit a similar inhibitory effect on neovascularization. Therefore, this example has confirmed that the miR-15a-5p seed sequence has a regulation and control effect on both sides.

TABLE 4

| Without | Name of | | Degree of inhibition of |
|---|---|---|---|
| mutation | sequence | Sequences | neovascularization (%) |
| WT sequence | WT | UAGCAGCACAUAAUGGUUUGUG | 74.8 ± 1.15 |
| | | (SEQ ID NO: 1) | |
| Number of | Name of | | Degree of inhibition of |
| mutation sites | sequence | Sequences | neovascularization (%) |
| 1 | M1 | UAGCAGCACAUGAUGGUUUGUG | 73.23 ± 1.42 |
| | | (SEQ ID NO: 12) | |
| 1 | M2 | UAGCAGCACAUAAUAGUUUGUG | 74.31 ± 1.07 |
| | | (SEQ ID NO: 13) | |
| 1 | M3 | UAGCAGCACAUAAUGGUUUAUG | 72.77 ± 1.19 |
| | | (SEQ ID NO: 14) | |
| 2 | M4 | UAGCAGCACAUAAUGGUUGAUG | 70.55 ± 1.35 |
| | | (SEQ ID NO: 15) | |
| 2 | M5 | UAGCAGCACAUAAUGGCAUGUG | 72.62 ± 1.02 |
| | | (SEQ ID NO: 16) | |
| 3 | M6 | UAGCAGCAAGAAAUGGUUUGUG | 73.11 ± 0.97 |
| | | (SEQ ID NO: 17) | |
| 3 | M7 | UAGCAGCACAUAAAAUUUUGUG | 72.73 ± 1.51 |
| | | (SEQ ID NO: 18) | |
| 5 | M8 | UAGCAGCACAUAAUGGUCCAGU | 70.91 ± 0.86 |
| | | (SEQ ID NO: 19) | |

The preferred embodiments of the present disclosure are described in detail above, which, however, are not intended to limit the present disclosure. Within the scope of the technical conception of the present disclosure, various simple modifications can be made to the embodiments of the present disclosure, all of which will fall within the protection scope of the present disclosure.

In addition, it should be noted that the various specific technical features described in the above specific embodiments can be combined in any suitable manner in which the features do not contradict each other. In order to avoid unnecessary repetition, such combinations will not be illustrated separately.

1.-32. (canceled)

33. A method for treating and/or preventing a fundus oculi disease, comprising administering to a subject in need thereof an effective amount of miRNA, miRNA mimic, modified miRNA, the vector comprising a nucleic acid to be transcribed into miRNA, wherein a nucleotide sequence of the miRNA comprises ACGACGAU (SEQ ID NO: 10).

34. The method according to claim 33, wherein the fundus oculi disease is selected from one or a combination of two or more of vitreous disease, retinopathy, optic neuropathy, or choroidopathy.

35. The method according to claim 33, wherein the fundus oculi disease is selected from one or a combination of two or more of retinopathy of prematurity, retinal neovascular disease, choroidal neovascular disease, or diabetic retinopathy.

36. The method according to claim 33, wherein the nucleotide sequence of the miRNA comprises SEQ ID NO: 10, and has 60%, 80%, 90%, 95%, or 99% or more identity to the nucleotide sequence set forth in SEQ ID NO: 1, or comprises a nucleotide sequence with substitution, deletion, or insertion of one or more nucleotides as compared to the nucleotide sequence set forth in SEQ ID NO: 1.

37. The method according to claim 33, wherein the nucleotide sequence of the miRNA is SEQ ID NO: 1.

38. The method according to claim 33, wherein the miRNA or the mimic thereof comprises a sense strand and an antisense strand, the sense strand comprising SEQ ID NO: 1, or having 60%, 80%, 90%, 95%, or 99% or more homology to the nucleotide sequence set forth in SEQ ID NO: 1, or comprising a nucleotide sequence with substitution, deletion, or insertion of one or more nucleotides as compared to the nucleotide sequence set forth in SEQ ID NO: 1; the antisense strand comprising SEQ ID NO: 6, or having 60%, 80%, 90%, 95%, or 99% or more homology to the nucleotide sequence set forth in SEQ ID NO: 6, or comprising a nucleotide sequence with substitution, deletion, or insertion of one or more nucleotides as compared to the nucleotide sequence set forth in SEQ ID NO: 6.

39. The method according to claim 33, wherein the modified miRNA comprises a modification on a base.

40. The method according to claim 39, wherein the modification on the base comprises one or a combination of two or more of a cholesterol modification at the 3′ end, two thio-backbone modifications at the 5′ end, four thio-backbone modifications at the 3′ end, or a full-chain methoxy modification.

41. The method according to claim 33, wherein the nucleic acid to be transcribed into miRNA comprises TAGCAGCA (SEQ ID NO: 11).

42. The method according to claim 33, wherein the nucleic acid to be transcribed into miRNA comprises SEQ ID NO: 11, and has 60%, 80%, 90%, 95%, or 99% or more identity to the nucleotide sequence set forth in SEQ ID NO: 9, or comprises a nucleotide sequence with substitution, deletion, or insertion of one or more nucleotides as compared to the nucleotide sequence set forth in SEQ ID NO: 9.

43. The method according to claim 33, wherein the nucleic acid to be transcribed into miRNA is SEQ ID NO: 9.

44. The method according to claim 33, wherein the vector is a viral vector or a non-viral vector.

45. The method according to claim 44, wherein the viral vector comprises one or a combination of two or more of lentivirus vector, retrovirus vector, adenovirus vector, adeno-associated virus vector, poxvirus vector, or herpesvirus vector.

46. The method according to claim 44, wherein the non-viral vector comprises one or a combination of two or more of liposome, lipid nanoparticle, polymer, polypeptide, antibody, aptamer, or N-acetylgalactosamine.

47. The method according to claim 33, wherein a site of the administration may be an intraocular space or cavity of the subject.

48. The method according to claim 47, wherein a site of the administration may be one or a combination of two or more of aqueous humor in the anterior chamber, suspensory ligament, ciliary body, internal ciliary body and muscle, lens or iris, vitreous body, retina, choroid, or optic nerve, and the like.

49. A vector comprising a nucleic acid to be transcribed into miRNA, wherein a nucleotide sequence of the miRNA comprises SEQ ID NO: 10.

50. The vector according to claim 49, wherein the nucleic acid to be transcribed into miRNA comprises TAGCAGCA (SEQ ID NO: 11).

51. The vector according to claim 49, wherein the nucleic acid to be transcribed into miRNA comprises SEQ ID NO: 11, and has 60%, 80%, 90%, 95%, or 99% or more identity to the nucleotide sequence set forth in SEQ ID NO: 9, or comprises a nucleotide sequence with substitution, deletion, or insertion of one or more nucleotides as compared to the nucleotide sequence set forth in SEQ ID NO: 9.

52. A medicament or a pharmaceutical composition, comprising miRNA or a mimic thereof, modified miRNA or the vector according to claim 49, and a pharmaceutically acceptable auxiliary material, wherein a nucleotide sequence of the miRNA comprises ACGACGAU (SEQ ID NO: 10).
